(19) United States
(12) Patent Application Publication
(10) Pub. No.: US 2026/0098868 A1
(43) Pub. Date: Apr. 9, 2026

(54) BIOMARKERS IN LONG-COVID-19

(71) Applicant: LONDON HEALTH SCIENCES CENTRE RESEARCH INC., London (CA)

(72) Inventor: Douglas FRASER, London (CA)

(73) Assignee: LONDON HEALTH SCIENCES CENTRE RESEARCH INC., London, ON (CA)

(21) Appl. No.: 19/115,765

(22) PCT Filed: Sep. 28, 2023
(86) PCT No.: PCT/CA2023/051292

§ 371 Date: Mar. 26, 2025

Related U.S. Application Data
(60) Provisional application No. 63/411,046, filed on Sep. 28, 2022.

(51) Int. Cl. G01N 33/68; C12Q 1/34

(52) U.S. Cl. CPC G01N 33/6893; C12Q 1/34; G01N 2333/4704; G01N 2333/4706; G01N 2333/521; G01N 2333/70578; G01N 2333/914; G01N 2800/26; G01N 2800/52

(57) ABSTRACT

A method of diagnosing long-COVID-19 in a patient, the method comprising: (a) obtaining a test sample from the patient, (b) performing one or more assays configured to detect a level of one or more biomarkers in the test sample, (c) comparing the level of the one or more proteins in the test sample with a healthy control reference value of said one or more proteins, wherein a change in the level of the one or more biomarkers in the test sample relative to the healthy control reference value of said one or more proteins is indicative of long-COVID-19 diagnosis, wherein the one or more proteins are selected from Table 3.

### TECHNICAL FIELD

This invention relates to blood biomarkers in long COVID-19 and to methods of diagnosis, prognosis, determining recovery and treatment of long COVID-19 using said blood biomarkers.

### BACKGROUND

Coronavirus disease 2019 (COVID-19) is caused by the highly transmissible severe acute respiratory syndrome coronavirus 2 (SARS-CoV-2) (1). SARS-CoV-2 binds the cell surface angiotensin-converting enzyme 2 (ACE2) receptor, resulting in cell entry and viral replication (2). An innate immune response follows SARS-CoV-2 infection (3) and includes increased interferons, tumor necrosis factor, bradykinin, serine proteases, soluble thrombomodulin and clot lysis times (4-8), all contributing to microvascular and thrombotic disease (9, 10). COVID-19 induces a wide range of disease severity, with hospitalized patients suffering an overall mortality rate of approximately 27% (11).

Survivors of COVID-19 often suffer diffuse symptoms that can persist for 2-7 months, referred to as “Long-COVID” (12-15). Several mechanisms have been proposed to explain the diffuse symptoms associated with Long-COVID, including the organ-specific expression of ACE-2 receptors that may predispose particular systems to greater tissue injury and prolonged healing (e.g., lung, heart, brain) (16), and/or microvascular endothelial dysfunction secondary to exacerbated inflammation and thrombotic mechanisms (9, 17). A lack of pathophysiological mechanisms and specific diagnostic markers has led some to question whether Long-COVID is a true disease entity (18). Given the ongoing debate associated with Long-COVID diagnoses, and the fact that only symptomatic treatments are available, two major priorities to optimize care of Long-COVID patients include early disease recognition with specific diagnostics, as well as, identification of molecular mechanisms for future targeted therapies.

Accurate diagnosis, prognosis, determination of recovery and targeted therapies for LONG-COVID are lacking, making the identification of biomarkers and pathophysiological mechanisms critical to optimize Long-COVID care.

### SUMMARY

In one embodiment, this disclosure relates to a method of diagnosing long-COVID-19 in a patient, the method comprising: (a) obtaining a test sample from the patient, (b) performing one or more assays configured to detect a level of one or more biomarkers in the test sample, (c) comparing the level of the one or more proteins in the test sample with a healthy control reference value of said one or more proteins, wherein a change in the level of the one or more biomarkers in the test sample relative to the healthy control reference value of said one or more proteins is indicative of long-COVID-19 diagnosis, wherein the one or more proteins are selected from Table 3.

In one embodiment of the method of diagnosing long-COVID, the one or more proteins are selected from CXCL5, AP3S2, MAX, PDLIM7, EDAR, LTA4H, CRACR2A, CXCL3, FRZB.

In another embodiment of the method of diagnosing long-COVID, the one or more proteins are selected from CXCL5, AP3SE, MAX, PDLIM7, and FRZB.

In another embodiment of the method of diagnosing long-COVID, the one or more assays is a proteomic assay.

In another embodiment of the method of diagnosing long-COVID, when the patient is diagnosed with long-COVID-19, the method further includes treating the patient with a long-COVID-19 therapy.

In another embodiment of the method of diagnosing long-COVID, the long-COVID-19 therapy comprises administering to the patient a treatment that promotes angiogenesis and/or administering to the patient accelerators of angiogenesis.

In another embodiment of the method of diagnosing long-COVID, the long-COVID-19 therapy comprises administering to the patient at least one of the proteins listed in Table 3 having its level lower than the level of said one or more biomarker in the healthy control reference value.

In another embodiment of the method of diagnosing long-COVID, the long-COVID-19 therapy comprises administering to the patient at least one of FRZB, a source of FRZB, FN1, a source of FN1, CKMT1A, a source of CKMT1A, CKMT1B, a source of CKMT1B, HS6ST1, a source of HS6ST1, BMP6, a source of BMP6, ADAMTS15, a source of ADAMTS15, ANGPTL2, a source or ANGPTL2, IFNLR1, C1QA, a source of C1QA, DRAXIN, a source of DRAXIN, ADAMTSL and/or a source of ADAMTSL4.

In another embodiment of the method of diagnosing long-COVID, the long-COVID-19 therapy comprises administering to the patient an agent that reduces the level of at least one of the proteins listed in Table 3 having its level higher than the level of said one or more proteins in the healthy control reference value.

In another embodiment of the method of diagnosing long-COVID, the long-COVID-19 therapy comprises administering to the patient an agent that reduces the levels of at least one of CXCL5, LTA4H, CXCL3, EDAR, MAX, PDLIM7, CRACR2A and AP3S2.

In another embodiment of the method of diagnosing long-COVID, the method further comprises (i) obtaining one or more recovery samples from the subject during the subject's treatment for long-COVID, and (ii) comparing the level of at least one of the proteins listed in Table 3 in the recovery samples to the level of said at least one protein in the test sample and to the level of said at least one protein in the healthy control reference value, wherein an approximation in the levels of the at least one protein in the one or more recovery samples towards the healthy control reference value for said at least one protein relative to the levels of the one or more biomarker obtained in the test sample is indicative of a normalization of the patient.

In another embodiment, this disclosure provides for a method of treating long-COVID-19 in a patient, the method comprising administering to the patient at least one of: (i) one or more of the proteins listed in Table 3 having a level in the long-COVID-19 column lower than the level of said one or more proteins in the Healthy and COVID column, and (ii) an agent that reduces the level of one or more of the proteins listed in Table 3 having a level in the long-COVID column of Table 3 higher than the level of said one or more proteins in the Healthy and COVID column of Table 3.

In one embodiment of the method of treating long-COVID-19 in a patient, the agent reduces the level of one or more of CXCL5, LTA4H, CXCL3, EDAR, MAX, PDLIM7, CRACR2A and AP3S2.

In another embodiment of the method of treating long-COVID-19 in a patient, the method comprises administering to the patient at least one of FRZB, a source of FRZB, FN1, a source of FN1, CKMT1A, a source of CKMT1A, CKMT1B, a source of CKMT1B, HS6ST1, a source of HS6ST1, BMP6, a source of BMP6, ADAMTS15, a source of ADAMTS15, ANGPTL2, a source or ANGPTL2, IFNLR1, C1QA, a source of C1QA, DRAXIN, a source of DRAXIN, ADAMTSL and/or a source of ADAMTSL4.

In another embodiment, the present disclosure provides for a use of an agent that reduces the level of at least one of the proteins listed in Table 3 having a level in the long-Long-COVID column lower than the level of said at least one protein in the Healthy and COVID column for the treatment of long-COVID-19. In one aspect, the agent reduces the level of at least one of CXCL5, LTA4H, CXCL3, EDAR, MAX, PDLIM7, CRACR2A and AP3S2.

In another embodiment, the present disclosure relates to a use of at least one protein listed in Table 3 or a source of said at least one protein listed in Table 3 having a level in the long-Long-COVID column higher than the level of said at least one protein in Healthy and COVID column for the treatment of long-COVID-19. In one aspect, the at least one protein listed in Table 3 or source of said at least one protein is FRZB, a source of FRZB, FN1, a source of FN1, CKMT1A, a source of CKMT1A, CKMT1B, a source of CKMT1B, HS6ST1, a source of HS6ST1, BMP6, a source of BMP6, ADAMTS15, a source of ADAMTS15, ANGPTL2, a source or ANGPTL2, IFNLR1, C1QA, a source of C1QA, DRAXIN, a source of DRAXIN, ADAMTSL and/or a source of ADAMTSL4. In another aspect the at least one protein listed in Table 3 or source of said at least one protein is FRZB and/or a source of FRZB.

### BRIEF DESCRIPTION OF THE DRAWINGS

The following figures illustrate various aspects and preferred and alternative embodiments of the disclosure.

FIGS. 1A to 1C. Cohorts plotted with tSNEs based on multiple protein combinations (protein combinations illustrated using feature reduction). 1A: Subjects plotted in two-dimensions, following t-SNE dimensionality reduction of all 119 important proteins (Table 3) determined by Boruta feature reduction, shows separation cluster of Long-COVID outpatients from acutely ill COVID-19 inpatients and healthy control subjects. 1B: Subjects plotted in two-dimensions, following t-SNE dimensionality reduction of top 9 important proteins (Table 4) determined by Recursive Feature Selection with 50% threshold, shows separation cluster of Long-COVID outpatients from acutely ill COVID-19 inpatients and healthy control subjects 1C: Subjects plotted in two-dimensions, following t-SNE dimensionality reduction of top 5 important proteins (CXCL5, AP3SE, MAX, PDLIM7, and FRZB) determined by Recursive Feature Selection with 80% threshold, shows separation cluster of Long-COVID outpatients from acutely ill COVID-19 inpatients and healthy control subjects with some mixing

FIGS. 2A to 2B. Heatmaps demonstrating the pairwise Eucladian Distance between cohort's biomarker profiles with respect to 2 protein combinations (lower distances between patients indicate similar biomarker profiles while larger distances indicate large differences between profiles, as indicated with the pseudocolor on the bar scale). 2A: A heatmap demonstrated the pairwise cosine similarity between cohort's protein profiles with respect to top 9 proteins (Table 4). Greater cosine similarity measure between subjects indicates similar protein profiles while smaller measure indicates large differences between profiles (distance was pseudocolored on the bar scale). The protein profile of Long-COVID outpatients is distinctively different from all other cohorts. 2B: A heatmap demonstrated the pairwise cosine similarity between cohort's protein profiles with respect to top 5 proteins (CXCL5, AP3SE, MAX, PDLIM7, and FRZB). Greater cosine similarity measure between subjects indicates similar protein profiles while smaller measure indicates large differences between profiles (distance was pseudocolored on the bar scale). The protein profile of Long-COVID outpatients is distinctively different from all other cohorts.

FIGS. 3A to 3I. Protein Expression of Optimal 9 Proteins in Long-COVID. Points are Long-COVID outpatient measurements; filled area represents 5th percentile to 95th percentile protein expression range of healthy control subjects. A-D, F-I Plots demonstrating elevated protein expression (Y axis) in Long-COVID compared to healthy controls versus time after acute infection for CXCL5, AP3S2, MAX, PDLIM7, EDAR, LTA4h, CRACR2A, CXCL3. E A plot demonstrating decreased FRZB expression in Long-COVID compared to healthy controls versus time after acute infection. In each of 3A to 3I: Y axis is protein expression and X axis is Days from COVID-19 Infection Onset.

FIGS. 4A to 4B. Frequency of protein expression in major organs/body systems and cell type. 4A: A bar plot demonstrating the percentage of proteins that are expressed in specific major organs and body systems determined by Natural Language Processing. There were total of 60 proteins out of the 119 proteins (50%) with UniProt organ system expression information. The organ system classification combines NLP identified organs, tissue, multi-level tissue and anatomical system entities. 4B A bar plot demonstrating the percentage of proteins that are expressed in specific cell types determined by Natural Language Processing. There were total of 44 proteins out of the 119 proteins (37%) with UniProt cell type expression information. Only those cell types with percentages greater than 5% are shown for visualization clarity.

FIG. 5. Recursive Feature Selection of 119 Protein Results after 10000 Runs. The plot showcases the number of Recursive Feature Selection runs each protein made it into the top 10. The 5000 run threshold (50%) is shown with the second dashed line from the top and the 8000 run threshold (80%) is shown with the first dashed line from the top. Out of the 119 proteins, only the shown molecules made it into the top 10 at least once.

### DESCRIPTION OF THE DISCLOSURE

### Abbreviations

Unless defined otherwise, all technical and scientific terms used herein have the same meaning as commonly understood by one of ordinary skill in the art to which this invention belongs. Also, unless indicated otherwise, except within the claims, the use of “or” includes “and” and vice versa. Non-limiting terms are not to be construed as limiting unless expressly stated or the context clearly indicates otherwise (for example “including”, “having” and “comprising” typically indicate “including without limitation”). Singular forms including in the claims such as “a”, “an” and “the” include the plural reference unless expressly stated otherwise. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

The contents of all documents (including patent documents and non-patent literature) cited in this application are incorporated herein by reference.

All numerical designations, e.g., levels, amounts and concentrations, including ranges, are approximations that typically may be varied (+) or (−) by increments of 0.1, 1.0, or 10.0, as appropriate. All numerical designations may be understood as preceded by the term “about”.

“COVID-19 subjects” are subjects who are confirmed SARS-COVID-19 positive on PCR testing. In the Examples, the studies have been carried out with ward COVID-19 subjects (mild/moderate disease) and with intensive care unit COVID-19 subjects (severe disease or critically ill).

“Healthy controls” are subjects without disease, acute illness, or prescription medications, and that were previously banked; samples were obtained prior to the emergence of SARS-CoV-2 and therefore considered negative for COVID-19.

“Long-COVID” refers to subjects not recovering for several weeks or months following the start of symptoms that were suggestive of COVID-19 and to survivors of COVID-19 that suffer diffuse symptoms that can persist for at least two months. According to the CDC website “Many post-COVID conditions can be improved through already established symptom management approaches (e.g., breathing exercises to improve symptoms of dyspnea). Creating a comprehensive rehabilitation plan may be helpful for some patients and might include physical and occupational therapy, speech and language therapy, vocational therapy, as well as neurologic rehabilitation for cognitive symptoms. A conservative physical rehabilitation plan might be indicated for some patients (e.g., persons with post-exertional malaise); consultation with physiatry for cautious initiation of exercise and recommendations about pacing may be useful. Gradual return to exercise as tolerated could be helpful for most patients. Optimizing management of underlying medical conditions might include counseling on lifestyle components such as nutrition, sleep, and stress reduction (e.g., meditation).” [Taken from CDC website: www.cdc.gov].

The term “level” covers either one or both levels of expression of a protein and/or levels of concentration of a protein.

The term “subject” as used herein refers all members of the animal kingdom including mammals, preferably humans.

The term “patient” as used herein refers to a subject that is long-COVID-19.

“Plasma” is the clear, straw-colored liquid portion of blood that remains after red blood cells, white blood cells, platelets and other cellular components are removed.

The term “pharmaceutically acceptable carrier”, “pharmaceutically acceptable excipient”, “physiologically acceptable carrier”, or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

Angiogenesis is a multistep process for the formation of new blood vessels. “Vaso-proliferative proteins” or “angiogenic proteins” refer to proteins that lead to activation of the cellular pathways that result in angiogenesis. Proteins, including angiogenic proteins, can be measured with antibody tests (i.e., Western blotting, Luminex bead-based assays, Proximity Extension Assay (PEA), planar multiplex assays, lateral flow assays, electrical conductivity devices, electrochemiluminescence, proximal extension assay with oligonucleotide-labeled antibodies, ELISA and RIA), flow cytometry or mass spec techniques. Enzymes can be measured with enzyme assays that measure either the consumption of a substrate or production of product over time. Differential expression profiles may have important diagnostic value, even in the absence of specifically identified proteins. Single protein spots can then be detected, for example, by immunoblotting, multiple spots or proteins using protein microarrays. The term “proteomic profile” is used to refer to a representation of the expression pattern of a plurality of proteins in a biological sample, e.g., a biological fluid at a given time. The proteomic profile can, for example, be represented as a mass spectrum, but other representations based on any physicochemical or biochemical properties of the proteins are also included. Thus, the proteomic profile may, for example, be based on differences in the electrophoretic properties of proteins, as determined by two-dimensional gel electrophoresis, e.g., by 2-D PAGE, and can be represented, e.g., as a plurality of spots in a two-dimensional electrophoresis gel. The proteomic profile typically represents or contains information that could range from a few peaks to a complex profile representing 50, 1,000 or more peaks. Thus, for example, the proteomic profile may contain or represent at least 2, or at least 5 or at least 10 or at least 15, or at least 20, or at least 25, or at least 30, or at least 35, or at least 40, or at least 45, or at least 50 proteins, or over 1,000 proteins.

Any suitable point-of-care measurement devices can be used to measure the proteins of the present disclosure, including testing with portable, table/counter-top, hand-held, lateral flow device (including lateral flow immunochromatographic assay), chip, aptamers (short, single-stranded DNA or RNA (ssDNA or ssRNA) molecules that can selectively bind to a specific target, including proteins, peptides, carbohydrates, small molecules, toxins, and even live cells), surface plasmon resonance or MS protein testing instruments.

The terms “agent”, “drug”, therapeutic agent”, “chemotherapeutic agent”, “active Ingredient”, “active compound”, and “active substance” refer to a compound, which is administered, alone or in combination with one or more pharmaceutically acceptable excipients or carriers, to a subject for treating, preventing, or ameliorating one or more symptoms of long-COVID pathology.

### Overview

The present disclosure relates to the use of at least one selected protein to accurately diagnose, prognose, follow for recovery and guide treatment of long-COVID in a subject. The present disclosure relates also to the use of at least one selected protein or inhibitor in the treatment of long-COVID.

### Diagnosis

The present disclosure relates to the diagnosis of long-COVID in a subject using the levels in a test sample taken from a subject of one or a combination of two or more (i.e., at least one) of the 119 proteins listed in Table 3. In another embodiment, the present disclosure relates to a method of diagnosis of long-COVID using the levels in a test sample taken from a subject of at least one of the 9 proteins listed in Table 4. In another embodiment, the present disclosure relates to a method of diagnosis of long-COVID using the levels in a test sample of at least one of CXCL5, AP3SE, MAX, PDLIM7, and/or FRZB.

In one embodiment, the present disclosure involves comparing the levels of at least one of the 119 proteins listed in Table 3 in a subject's sample, such as blood, blood plasma, blood serum, capillary blood/plasma, venous blood, saliva, synovial fluid, urine, spinal fluid, bronchoalveolar lavage, sweat, tears, breath samples and extracts (for example extracts of hippocampal tissue or ipsilateral cortex tissue), using quantitative measurements of said at least one protein, to a known reference range or cut-off value, or to the levels of said at least one protein in a library of measurements of known COVID-19 patients or to a library of healthy subjects. A change (i.e., a drop or an increase) in the level of the at least one protein listed in Table 3 in the subject's sample relative to the known reference range, cut-off value or libraries being indicative of the subject having long-COVID. In one embodiment the change is a drop or decrease in the level of the at least one protein. In another embodiment the change is an increase in the level of the at least one protein.

In one embodiment, the at least one protein is at least one of CXCL5, AP3S2, MAX, PDLIM7, EDAR, LTA4H, CRACR2A, CXCL3 and FRZB. In another the at least one protein is at least one of CXCL5, AP3SE, MAX, PDLIM7, and FRZB.

A list of 119 proteins that are statistically significant for diagnosing long-COVID are listed in Table 3. As illustrated in Table 3, each of the 119 proteins has AUC between 0.91 to 1.0. As such, any one of the 119 proteins of Table 3 is useful for diagnosis of long-COVID in a subject, and each one of the 119 proteins listed in Table 3 is also useful for prognosticating and/or following up treatment or disease resolution of Long-COVID.

A library of measurements of the proteins listed in Table 3 may be established for diagnosed COVID-19 cases and for healthy control subjects. A comparison may be made of the subject's protein measurements against the libraries of long-COVID and the libraries of healthy control subjects (referred to also as negative control or normal control) to determine if the patient long-COVID.

The libraries may be provided in a computer product (memory sticks, as an app for handheld devices such as tablets, pads, smart watches, cellular phones and so forth), or they may be uploaded to the cloud, the memory of a computer system, including main frames, desktops, laptops, handheld devices such as tablets, pads, smart watches and cellular phones. Blood or any other bodily fluid, for example whole blood, blood plasma, blood serum, capillary blood sample, saliva, synovial fluid, urine, spinal fluid, bronchoalveolar lavage, tears, sweat, extracts, breath sample and so forth, may be taken from a subject suspected of having long-COVID and the level of the at least one protein measured and compared to the library.

In one embodiment of this disclosure, once a subject is diagnosed with long-COVID, the method further includes treating the patient for long-COVID.

Treatment may include one of the treatment methods of the present disclosure, or standard treatments for long-COVID, including therapeutic exercise, physiotherapy, oxygen, inhalers (salbutamol, steroids), nasal sprays and/or anticoagulation.

In another embodiment, the present disclosure provides a method to determine the recovery of a long-COVID patient. In one embodiment the method comprises obtaining one or more recovery samples from the long-COVID patient during the patient's treatment for long-COVID, (b) comparing the levels of at least one of the proteins listed in Table 3 in the recovery samples to the levels of said at least one protein in the test sample obtained from the patient when he/she was diagnosed for long-COVID and to the levels of said at least one protein in a reference healthy control. Approximation in the levels of the at least one protein in the one or more recovery samples towards the reference healthy control of said at least one protein relative to the levels of the one or more biomarker obtained in the test sample is indicative of a normalization of the patient.

### Treatment

In one embodiment, treatment of long-COVID includes target therapy comprising administering to the patient a protein (or combination of proteins), including sources of said protein, listed in Table 3 having a level in a long-COVID-19 test sample lower than the level of said protein in a healthy control subject or than the level of said protein in a COVID-19 subject.

In another embodiment, treatment of long-COVID includes administering to a long-COVID patient at least one of FRZB, a source of FRZB, FN1, a source of FN1, CKMT1A, a source of CKMT1A, CKMT1B, a source of CKMT1B, HS6ST1, a source of HS6ST1, BMP6, a source of BMP6, ADAMTS15, a source of ADAMTS15, ANGPTL2, a source or ANGPTL2, IFNLR1, C1QA, a source of C1QA, DRAXIN, a source of DRAXIN, ADAMTSL and/or a source of ADAMTSL4.

In another embodiment, treatment of long-COVID includes administering to a long-COVID patient FRZB or a source of FRZB.

In one embodiment, treatment of long-COVID includes target therapy comprising administering to the patient an agent that reduces the level of a protein listed in Table 3 having a level that is higher than the level of said biomarker in a healthy control subject and/or in a COVID-19 subject. In one embodiment, treatment of long-COVID includes administering to the long-COVID patient an agent that lowers the level of at least one of CXCL5, AP3S2, MAX, PDLIM7, EDAR, LTA4H, CRACR2A and/or CXCL3.

In one embodiment, the agent that reduces the level of a protein include agents that inhibit the synthesis or expression of the protein. Examples of agents include siRNA, antibodies/ligands that block expression of the proteins, and so forth.

In another embodiment, the agent that reduces the level of a protein is an agent that inactivates the protein, such as denatures or nicks the protein (i.e., restriction enzymes).

In another embodiment, treatment of long-COVID includes combining the therapies of the present disclosure with the administration to the patient of an accelerator of angiogenesis to encourage the angiogenesis process to be faster and complete.

In another embodiment, the treatments of the present disclosure are combined with standard symptomatic treatments for Long-COVID, including therapeutic exercise, physiotherapy, oxygen, inhalers (salbutamol, steroids), nasal sprays and/or anticoagulation.

Depending on the symptoms, treatment of long-COVID can include one or more interventions of the administration described above.

C-X-C motif chemokine 5 (CXCL5 or ENA78) is a protein that in humans is encoded by the CXCL5 gene. Diseases associated with CXCL5 include Nonspecific Interstitial Pneumonia and Pulmonary Sarcoidosis. This small molecule peptide, interacts with GPCRs to attract neutrophils during inflammation processes. It has also been identified to participate in angiogenesis, tumor growth, and metastasis CXCL5 has been linked to various cancers as a promotor and in inflammatory diseases. OLINK Panel: Cardiometabolic.

Leukotriene-A4 hydrolase (LTA4H) has two functions: converting LTA4 into LTB4, a neutrophil chemoattractant, and aminopeptidase activity. The proinflammatory role of LTA4H through LTB4 activity allows for LTA4H to be an anti-inflammatory target. LTA4H is also proposed as a cancer therapeutic target as LTA4H as it is found to be overexpressed in cancer. OLINK Panel: Oncology.

CXC Motif Chemokine Ligand 3 (CXCL3) is part of the CXC chemokine family. CXCL3 may play a role in acute inflammation as it is noted to activate neutrophils, basophils, eosinophils, monocytes, smooth muscle cells, and lymphocytes. CXCL3 has been shown to facilitate adipogenesis and plays a role in various cancers. OLINK Panel: Inflammation.

Ectodysplasin A receptor (EDAR) interacts with ectodysplasin A as a cell surface receptor that is critical for cell signaling and developmental pathways. EDAR has been associated with ectodermal dysplasia resulting in errors or differences in hair, teeth, and exocrine gland development. OLINK Panel: Inflammation.

MAX (also known as myc-associated factor X) is a transcription regulator and is critical to oncoprotein MYC function (15) which is associated with cell growth. As such MAX is a potential target for cancer drugs to inhibit MYC function. Mutations of MAX are proposed to cause hereditary pheochromocytoma, a type of neuroendocrine tumor consisting of neural crest cells localized in the adrenal medulla. MAX is also associated with regulating clock gene expression, part of the circadian clock. OLINK Panel: Neurology.

The PDZ and LIM domain protein 7 (PDLIM7, Enigma) binds to protein kinases using the LIM domain and actin filaments via the PDZ domain. PDLIM7 in mise is primarily found in actin-rich structures like the heart and vascular smooth muscle. PDLIM7 has been connected to vascular and heart development and is also linked to skeletal muscle development. OLINK Panel: Inflammation.

CRACR2A (Calcium Release Activated Channel Regulator 2A). Calcium release-activated channel regulator 2A (CRACR2A) is a conserved protein expressed in T cells. CRACR2A participates in T cell activation and regulation of endocytic traffic via dynein. Changes in CRACR2A functioning have been linked to immunodeficiency disorders. OLINK Panel: Oncology.

AP-3 complex subunit sigma-2 (AP3S2). Adapter-related protein complex 3 subunit sigma-2 (AP3S2) is a small chain of the clathrin-based Adapterrelated heterotetramer protein complex 3 (AP-3). Two forms of AP-3 with different chain variations exist, a ubiquitous AP-3 and a brain-specific AP-3, both of which have AP3S2 chains. AP-3 in mammals is linked to the lysosome and lysosome-related organelles as well as neurotransmitter release mechanisms. SNPs in the AP3S2 have been associated with type 2 diabetes mellitus in Chinese and South Asian populations. Defects in other neuron AP-3 subunits have shown to lead to severe neurological abnormalities including neurodevelopmental delays, intellectual disability and seizures. OLINK Panel: Cardiometabolic.

Frzb is a Wnt-binding protein especially important in embryonic development. The protein is encoded by the FRZB (Frizzled Related Protein) gene. Diseases associated with FRZB include Osteoarthritis and Holzgreve Syndrome.

### Therapeutic Angiogenesis

1. Therapeutic Angiogenic Drugs (Li V W, Kung E F, Li W W. Molecular Therapy for Wounds: Modalities for stimulating Angiogenesis and Granulation. Manual of Wound Management (Bok Lec, Editor) McGraw Hill, 2004, p. 17-43; Li W, Talcott K, Zhai A, Kruger E, Li V. The Role of Therapeutic Angiogenesis in Tissue Repair and Regeneration Adv Skin Wound Care 2005; 18:491-500; Smiell J M, Wieman T J, Steed D L, et al. Efficacy and safety of becaplermin (recombinant human platelet-derived growth factor-BB) in patients with nonhealing, lower extremity diabetic ulcers: a combined analysis of four randomized studies. Wound Repair Regen. 1999; 7:335-346): Growth factor-based therapies include the only FDA-approved recombinant protein drug recombinant human Platelet-derived growth factor (rhPDGF) (becaplermin, REGRANEX® 0.01% gel), which is indicated for diabetic neuropathic lower extremity ulcers.
Growth factors can also be delivered through autologous isolates of patient platelets such as Autologel, SmartPReP.
Currently, there are no FDA-approved angiogenic drugs for the treatment of ischemic cardiovascular disease.
Some early-stage clinical trials of therapeutic angiogenic agents have demonstrated reductions in symptoms of angina, increase in ability to exercise, and objective evidence of improved perfusion and left ventricular function following therapy.
Therapeutic Angiogenesis Promoting Devices: Negative pressure wound therapy (NPWT) such as the Vacuum Assisted Closure (V.A.C.) system induces angiogenesis through tissue microdeformations and mechanochemical coupling and signal transduction; MIST ultrasound is a low-frequency and low-intensity non-contact device that results in cell stimulation and increased wound perfusion; Hyperbaric Oxygen (HBO) promotes angiogenesis and wound healing by increasing Vascular endothelial growth factor (VEGF) expression and recruiting endothelial progenitor cells.

### Cell-Based Therapies:

Tissue engineered products approved by the FDA include the bilayered skin substitute Grafstkin (Apligraf®) and the fibroblast dermal skin substitute Dermagraft. These products contain living or cryopreserved cells on a matrix capable of secreting and releasing multiple angiogenic growth factors into the wound bed.
CD34+ endothelial progenitor cells (EPC) derived from bone marrow or from peripheral blood have been found to enhance angiogenesis in ischemic tissues, increase transcutaneous oxygen, improve ankle-brachial index (ABI), increase collateral vessels by angiography and improve healing of leg ulcers.
Integra® Dermal Regeneration Template is an advanced skin replacement matrix that consists of a complex three-dimensional porous matrix that acts as a scaffold for cell migration and allows for regeneration of the dermal layer of the patient's skin. It can be used for Diabetic Foot Ulcers.

2. VEGF/VPF (Tan, Q., et al., European Journal of Cardio-thoracic Surgery 31 (2007) 806-811; vascular permeability factor (VPF).

3. ANG1/TIE1 pathway—ANG1 has vasculoprotective effects. It enhances the stability of newly formed vessels, inhibits vascular permeability induced by several inflammatory cytokines and attenuates pathological responses, including fibrosis. Recently, the angiopoietin (ANG)-TIE signaling pathway has emerged as an attractive vascular drug target. The ANG-TIE pathway is required for lymphatic and blood vessel development.
1. Recombinant/viral vectors ANG1 (Angiopoietin 1)
2. ANG2 (Angiopoietin 2) inhibitors (ANG2 blocks angiogenesis)

### 4. Statin Therapy

Low-dose statin therapy may promote angiogenesis via multiple mechanisms, including enhanced NO production, augmented VEGF release, and activation of the Akt signaling pathway

### 5. Therapeutic/Prescribed Exercise

Exercise stimulates angiogenesis in skeletal muscle and heart. A lack of exercise leads to capillary regression.

### 6. Plasminogen Activator System (Plasmin)

https://www.news-medical.net/health/Angiogenesis-Stimulation.aspx Plasmin also activates Matrix metalloproteinases (MMPs) such as MMP-1, MMP-3, and MMP-9. These are metalloproteinases.

### 6. Growth Factors (Fibroblast Growth Factor 2 (FGF2) and VEGF)

FGF 2 is vital for angiogenesis. It induces multiplication and movement of the cells as well as uPA production by endothelial cells. FGF-2 induces tube formation in collagen gels and alters integrin expression that helps in angiogenesis.
7. Combination therapies—VEGF, FGF, MMP1, plasma, etc. (Sabra, M., et al., Int. J. Mol. Sci. 2021, 22, 3722)
8. Stimulators of angiogenesis: VEGF, FGF, Hepatocyte Growth Factor (HGF), Angiopoietin 1 (Ang1) and Angiopoietin 2 (Ang2), Platelet-derived growth factors (PDGFs), insulin-like growth factor (IGF), Endoglin Interleukin 8, Thyroxin, VE-cadherin, Granulocyte colony-stimulating factor (G-CSF), Integrins, Ephrin, Endothelial nitric oxide synthase (eNOS), Transforming growth factor beta (TGFbeta), YKL40, HIF1α (Hypoxia Inducible Factor 1 Subunit Alpha), HDGF (Heparin Binding Growth Factor), Notch/DLL4 (Delta-like 4), Semaphrorins.
9. Chinese herbal medicines. Chinese herbal medicines that target angiogenesis can provide therapeutic effect, including active components Salvianolic acid A, Tanshinone IIA, Ferulaic acid, Rhodiola, Salidroside, Astragalosides, Berberine, Puerarin and Extract of Geum japonicum. These active compotnets can be obtained from Radix Salvia miltiorrhiza, Radix Angelica Sinensis, Rhizoma Rhodiolae Kirilowii, Shanxi Astragalus membranaceus, Berberis and Berberis aristate, Radix Puerariae, Germ japonicum (Dongqing Guo, et al., Frontiers in Pharmacology, (2018) 9, 428).

Compositions of the present disclosure comprising one or more of the biomarkers of Table 3 of the present disclosure or inhibitor or antagonist as the case may be, include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration. The compositions may conveniently be presented in unit dosage form and may be prepared by any of the methods well known in the art of pharmacy.

Formulations of the proteins listed in Table 3 suitable for oral administration may be presented as discrete units such as capsules, cachets or tablets each containing a predetermined amount of the protein (i.e., the active ingredient); as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol.

The compositions and/or pharmaceutical preparations may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

In addition to the formulations described previously, the compounds may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose.

For administration by inhalation, compounds may be delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the disclosure may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

The amount of active ingredient that may be combined with the carrier materials to produce a single dosage form will vary depending upon the host treated and the particular mode of administration.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

All of the treatments described in this disclosure can be administered alone or in combination.

In order to aid in the understanding and preparation of the within invention, the following illustrative, non-limiting, examples are provided.

### Example 1

### Methods

### Study Participants and Blood Sampling

All patients were screened and enrolled from our tertiary care system (London, Ontario, Canada). Both Long-COVID and acutely ill COVID-19 had their COVID-19 status confirmed as part of standard hospital testing by detection of two SARS-CoV-2 viral genes using polymerase chain reaction (CDC 2019-Novel Coronavirus 2019). Long-COVID outpatients had been referred to a specialty clinic based on prolonged, diffuse symptoms. Venous blood was drawn once as part of a larger clinical screen, and excess plasma collected for later research analysis by Pathology and Laboratory Medicine (PaLM). Both Ward and intensive care unit (ICU) patients were enrolled on admission to the hospital. Blood sampling for inpatients began on admission, Ward or ICU Day 1. Daily blood was obtained from critically ill ICU patients via indwelling catheters and if a venipuncture was required, research blood draws were coordinated with a clinically indicated blood draw. In keeping with accepted research phlebotomy protocols for adult patients, blood draws did not exceed maximal volumes (NIH Hrpp 2009 (19)). Blood was centrifuged and plasma isolated, aliquoted at 250 μL, and frozen at −80° C. All samples remained frozen until use and freeze/thaw cycles were avoided. The healthy control subjects were individuals without disease, acute illness, or prescription medications that were previously banked in the Translational Research Centre, London, ON (Brisson et al. 2012 (20); Gillio-Meina et al. 2013 (21). These latter samples were obtained prior to the emergence of SARS-CoV-2 in our region and therefore, were considered to not have been exposed to the virus.

### Patient Demographics, Clinical Data, and Cohort Matching

Baseline characteristics for Long-COVID, Ward and, ICU patients were recorded and included age, sex, comorbidities, presenting symptoms, interventions, and laboratory measurements. For Long-COVID patients, we recorded both initial infection variables and clinical variables at follow-up clinic visit. For the latter, we focused on lingering symptoms, laboratory values and interventions. For ICU patients, we included standard illness severity scores, including Multiple Organ Dysfunction Score (MODS) (Priestap et al. 2020; (22)) and Sequential Organ Failure Assessment scores (Singer et al. 2016 (23). The PaO₂ to FiO₂ ratio and chest radiograph findings were recorded for all ICU patients. We also recorded clinical interventions received during the observation period including the use of antibiotics, antiviral agents, systemic corticosteroids, vasoactive medications, venous thromboembolism prophylaxis, antiplatelet, or anticoagulation treatment, renal replacement therapy, high flow oxygen therapy, and mechanical ventilation (invasive and noninvasive). Final participant groups were constructed by age- and sex-matching Long-COVID outpatients with Ward COVID-19 inpatients, ICU COVID-19 inpatients, and healthy control subjects.

### Proximity Extension Assay

Plasma was thawed for PEA testing (Olink Proteomics, Sweden) as previously described (Lundberg et al. 2011 (24); Assarsson et al. 2014 (25)). Specifically, we measured a total of 3072 plasma proteins in the plasma of Long-COVID, acutely ill COVID-19, and healthy control subjects. The Olink Explore 3072 library consists of multiple panels with some duplicated proteins leading to the measurement of 2925 unique proteins. The PEA was performed in three steps: (1) antibody pairs, labeled with unique DNA oligonucleotides, were attached to their target antigen in plasma; (2) oligonucleotides that were brought into proximity hybridized and were extended by a DNA polymerase; and (3) the newly formed DNA barcode was amplified for high-sensitivity, high-specificity readout with next generation sequencing (NovaSeq Platform; Illumina Inc., San Diego, CA). Data were generated and expressed as relative quantification on the log 2 scale of normalized protein expression (NPX) values. Data were converted from log 2 scale to normal scale to better represent protein expression. Samples were screened based on quality controls for immunoassay and detection, as well as degree of hemolysis. Following proteomic quality control, all 88 (22 healthy control, 22 Ward COVID-19, 22 ICU COVID-19, and 22 Long-COVID) patients/subjects were deemed suitable for analysis.

### Conventional Statistics

Patient baseline clinical characteristics were reported as median (IQRs) for continuous variables and frequency (%) for categorical variables. The individual biomarkers of Long-COVID outpatients were compared to a combined group of healthy controls, Ward COVID-19 inpatients, and ICU COVID-19 inpatients, using a Mann-Whitney U Test. A Kruskal-Wallis H-test for independent samples followed by a pairwise posthoc Dunn test was also conducted for the optimal models. A Bonferroni correction was applied to avoid multiple comparison complications, with only Bonferroni-corrected P-values being reported and those with a P<0.01 were considered to be statistically significant.

### Machine Learning

For machine learning, a Random Forest classifier based on decision trees was used to classify the Long-COVID cohort in comparison to a combined cohort of acutely ill COVID-19 ward/ICU inpatients and healthy control subjects by their biomarkers. The Boruta feature reduction algorithm was used to identify the most important biomarkers (Kursa and Rudnicki 2010). The Boruta algorithm is based on Random Forest classifiers and individually compares each biomarker to randomly generated data to determine if the biomarker is better at classifying than chance. The results from the Boruta feature reduction identified the most relevant biomarkers for classifying Long-COVID.

The following steps were undertaken to conduct a conservative analysis that mitigates concerns of relatively small sample sizes and overfitting due to Boruta feature reduction being based on Random Forest classifiers. First, the data was split into a feature reduction dataset (70%) and a testing dataset (30%), stratified by subject groups. The Boruta algorithm was run on the feature reduction dataset to determine the most relevant features. A reduced dataset was created from the testing dataset and only contained the most relevant features. The reduced dataset was then used for the classification of Long-COVID. To reduce overfitting and maintain a conservative model, three-fold cross-validation with a Random Forest of 10 trees and a maximum depth of three was used (Tang et al. 2018).

To prepare an optimal model, recursive feature elimination (RFE) was used. As a Random Forest is a set of decision trees, we were able to interrogate this collection of trees to identify the features that have the highest predictive value (viz., those features that frequently appear near the top of the decision tree). Based on this characteristic, RFE starts with the reduced dataset, fits a Random Forest classifier and determines the importance rankings. The algorithm then drops the least important feature and repeats the process until only 10 features are remaining. Due to the randomness in the algorithm and Random Forest models, 10,000 runs of RFE were conducted. Those features that were in the top 10 for more than a specified threshold of the 10,000 runs were determined to be the optimal features. The specified threshold is determined after the inspection of the RFE results. An optimal dataset containing only these optimal features was generated from the reduced dataset. The same classification process used for the reduced dataset was used on the optimal dataset.

Receiver operating characteristic (ROC) curves using Logistic Regression were conducted to determine the sensitivity and specificity of individual molecules for predicting Long-COVID status in comparison to healthy controls and COVID-19 patients. Area-under-the-curve (AUC) was calculated as an aggregate measure of protein performance across all possible classification thresholds (Bradley 1997 (26)). Precision and Recall were determined, including their combined metric (F1 score), which was calculated as the harmonic mean. A high F1 score indicated that both, Precision and Recall were high. The biomarker data was visualized with a nonlinear dimensionality reduction on the full, reduced, and optimal datasets using the t-distributed stochastic nearest neighbour embedding (t-SNE) algorithm. t-SNE assumes that the ‘optimal’ representation of the data lies on a manifold with complex geometry, but a low dimension, embedded in the full-dimensional space of the raw data (Van der Maaten and Hinton 2008 (27)). A pairwise comparison, using cosine similarity, was conducted to determine the similarity between subjects across the selected biomarkers (Jambu 1991 (28)). As such, subjects similar across their selected biomarker profile have a score closer to 1, while dissimilar subjects have a score closer to 0. The analysis was done with data Min-Max scaled between 0 and 1 and the cosine similarities were visualized using a heatmap. The machine learning analysis was conducted using Python version 3.9.7 and Scikit-Learn version 1.0.2 (Pedregosa et al. 2011 (29)).

### Natural Language Processing

Exploratory expression analysis was also conducted to determine physiological areas of interest in Long-COVID subjects. Protein expression tissue specificity was parsed from UniProt Knowledgebase using the UniProt website REST API (Bateman et al. 2021 (30)). The tissue specificity was unstructured text on the expression at the mRNA or protein level in cells or tissues gathered manually by experts. The expression information was processed by Natural Language Processing (NLP) using the Stanza python package implemented with spaCy (Python v. 3.10.4; spaCy v. 3.3.1; spaCy-Stanza v. 1.0.2; negspaCy v. 1.0.3) (Zhang et al. 2021a (31); Qi et al. 2020 (32); Honnibal et al. 2020 (33)). An NLP named-entity recognition (NER) pipeline was configured with the MIMIC package for preprocessing, negation detection, and the pretrained Stanza BioNLP13CG Biomedical model. The negation detection was done using the NegEx-based negspaCy implementation with a modified English clinical term set to filter negative expression terms. Although the BioNLP13CG biomedical model was based on Cancer Genetics and publicly available PubMed abstracts, in comparison to the other Stanza models, it provided the most granular entity classification, including anatomical system, organ, tissue, multi-level tissue, and cell type entities. The detected organ and cell type entities were manually classified into keyword-based groups separately. The manual expression curation process relies on existing literature and is not easily structured into specific organ systems. To include the maximum expression information in the analysis, the organ, tissue, multi-tissue, and anatomical system entity types were combined and manually sorted into organ systems. The frequency of the keyword-based categories with respect to the relevant proteins was determined to identify physiological patterns of expression.

### Results

A total of 4 age- and sex-matched groups were included consisting of Long-COVID outpatients (median years old=61; IQR=21; n=22), Ward COVID-19 inpatients (median years old=60; IQR=22; n=22), ICU COVID-19 inpatients (median years old=58; IQR=18; n=22) and healthy control subjects (median years old=59; IQR=16; n=22). There were no significant differences with regards to age (Kruskal-Wallis H-test, P=0.9880) and sex (Chi-Square, P=1.00) between the 4 cohorts. Baseline demographic characteristics, comorbidities, laboratory measurements, interventions, and chest x-ray findings of Long-COVID outpatients and the Ward/ICU COVID-19 inpatients, are reported in Table 1 and Table 2 respectively. Long-COVID outpatients had a single blood draw at their clinic visit, whereas blood from Ward and ICU COVID-19 inpatients was drawn on day 1 of admission. Long-COVID patients had significantly elevated lymphocyte measurements in comparison to both Ward and ICU COVID-19 patients as determined by a Kruskal-Wallis H-test (p<0.0001). The mortality rates for Ward and ICU COVID-19 inpatients were 9.1% and 45.5%, respectively.

A total of 2,943 unique blood biomarkers were identified and measured using Proximity Extension Assay technology following the removal of duplicates.

Proximity extension assay (PEA) is a form of targeted proteomics that combines protein-specific antibodies with unique deoxyribonucleic acid (DNA) tags, followed by amplification with either quantitative polymerase chain reaction or next generation sequencing. The result is a targeted proteomic approach to biomarker discovery with excellent sensitivity and specificity over a broad dynamic range. Indeed, PEA offers a high level of precision, and is well-suited for large-scale studies due to minimal matrix interference and cross reactivity. Indeed, conventional immunoassays suffer from cross-reactivity due to unspecific binding of antibodies, whereas, the unique DNA oligonucleotide sequences used in PEA result in matched DNA-pairs that greatly diminish non-specific antibody binding.

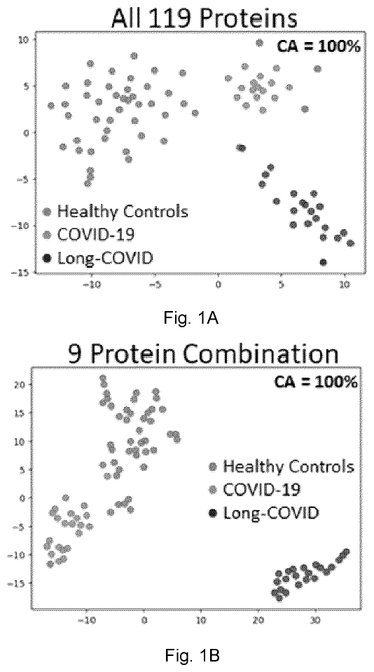

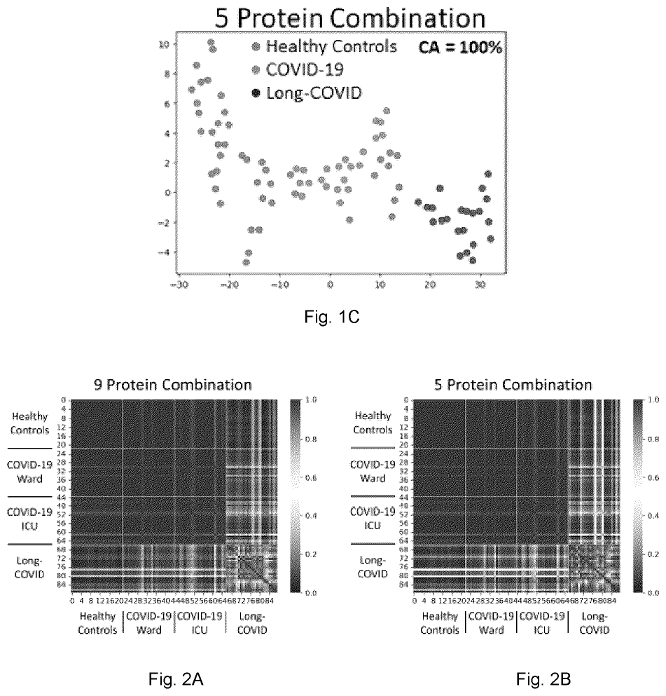

The PEA was performed in three steps: (1) antibody pairs, labeled with unique DNA oligonucleotides, were attached to their target antigen in plasma; (2) oligonucleotides that were brought into proximity hybridized and were extended by a DNA polymerase; and (3) the newly formed DNA barcode was amplified for high-sensitivity, high-specificity readout with next generation sequencing (NovaSeq Platform; Illumina Inc., San Diego, CA). Individual samples were screened based on quality controls for immunoassay and detection, as well as degree of hemolysis. Intraassay variability was minimized via robotic pipetting for volume accuracy, and through normalization using three specifically engineered internal controls that were added to each sample; including one control for the incubation, one for the extension and one for the amplification. External negative control and plate control samples were included in each sample plate in triplicate to improve inter-assay precision. Following proteomic quality control, plasma measurements from all 35 participants were deemed suitable for analysis. The data generated were expressed as relative quantification on the log 2 scale of normalized protein expression (NPX) values. NPX values were rank-based normal transformed for further analyses Boruta feature reduction identified 119 biomarkers to be relevant in classifying Long-COVID patients when compared to a combined cohort of COVID-19 and healthy control subjects. All 119 relevant biomarkers were significantly different between Long-COVID subjects, and the other subjects as calculated by Mann-Whitney U test with Bonferroni multiple-comparison correction (corrected P<0.0001; Table 3). Of the 119 biomarkers, only 10 exhibited decreased expression (FRZB, FN1, CKMT1A_CKMT1B, HS6ST1, BMP6, ANGPTL2, IFNLR1, C1QA, DRAXIN, and ADAMTSL4). Each of the 119 relevant biomarkers had excellent individual classification ability with Area-Under-the-Curves ranging between 0.91 and 1.00. Using the 119 relevant blood biomarkers, a t-SNE plot illustrated that Long-COVID patients were easily separable from acutely ill COVID-19 inpatients and healthy control subjects (FIG. 1A).

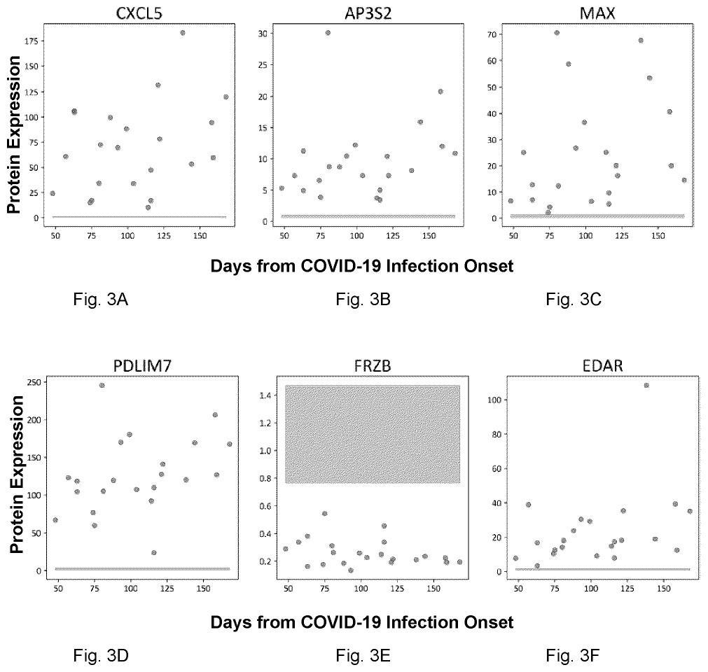

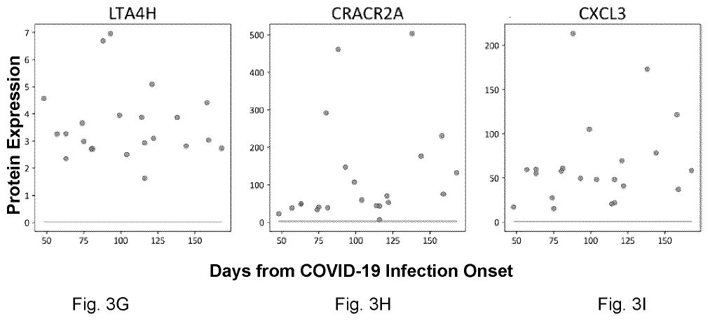

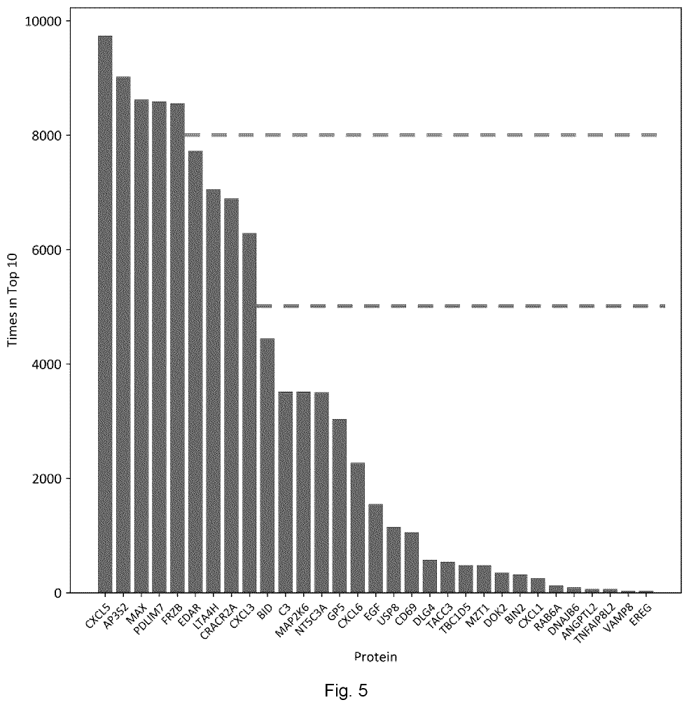

Recursive feature elimination was used to determine two sets of optimal biomarkers, one with a threshold of 50% and another with a threshold of 80% (FIG. 5). The threshold represents the percentage of runs, out of 10,000 RFE repetitions, that a particular protein was in the top 10 reduced proteins. With the threshold of 50%, an optimal set of nine proteins (CXCL5, AP3S2, MAX, PDLIM7, EDAR, LTA4H, CRACR2A, CXCL3, FRZB) was determined from the 119 relevant proteins (see Table 4). A t-SNE plot based on the nine optimal biomarkers showcases a distinct separation between the Long-COVID cohort and the combined healthy and COVID-19 subjects (FIG. 1B, AUC 1.00, classification accuracy 100%). With a threshold of 80%, an optimal set of five proteins (CXCL5, AP3SE, MAX, PDLIM7, FRZB) was determined from the 119 relevant proteins. A t-SNE plot based on the five optimal biomarkers showcases a distinct separation between the Long-COVID cohort and the combined healthy and COVID-19 subjects (FIG. 1C, AUC 1.00, classification accuracy 100%). All optimal biomarkers had excellent individual classification ability with an AUC of 1.00, except for CRACR2A which had an AUC of 0.97. All of the nine optimal proteins were significantly elevated in Long-COVID outpatients, other than FRZB, which was significantly decreased in Long-COVID outpatients (FIGS. 3A-3I and Table 4). Confounding variables, such as steroid administration, were excluded via correlation analysis between patient/subject variables and protein expression (data not shown).

Pairwise cosine similarity between all subjects was calculated to compare the cohorts in terms of a holistic nine and five optimal protein profile is presented in FIG. 2A and FIG. 2B, respectively. For both nine and five optimal protein set, the protein profile between the healthy control, Ward COVID-19 and ICU COVID-19 was homogeneous. The Long-COVID subjects were relatively less homogeneous but distinct from the other 3 cohorts.

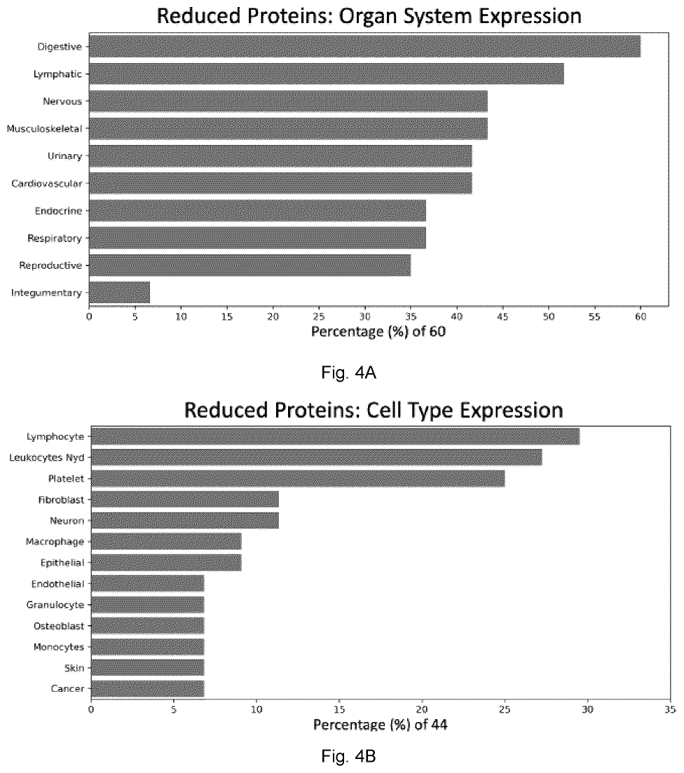

Named-entity recognition was conducted on the tissue expression information provided by the UniProt Knowledgebase. Out of the 119 reduced proteins, 60 (50.4%) had organ expression information (Table 5) and 44 (37.0%) had cell type expression information (Table 6). The percentage of the 60 molecules that are expressed in specific organ systems and the percentage of the 44 molecules that are expressed in specific cell types are presented in FIGS. 4A, 4B respectively. The leading organ system based on the number of changed proteins was the digestive system. Analyses of cell type expression demonstrated that the number of changed proteins was greatest in lymphocytes/leukocytes not yet determined.

TABLE 1
Long-COVID Outpatient Demographics and Clinical Data

| | Outpatients | |
|---|---|---|
| | (n = 22) | |
| Initial Infection Variable | | |
| Age (yrs), median (IQR) | 61.0 | (20.5) |
| Male sex, no. (%) | 12 | (54.5) |
| Diagnostic test: PCR, serology, no. (%) | 22 | (100.0) |
| Vaccination status at infection, no. (%) | 2 | (9.1) |
| Hospitalization, no. (%) | | |
| Ward | 7 | (30.4) |
| ICU | 1 | (4.3) |
| Comorbidities, no. (%) | | |
| Diabetes | 6 | (27.3) |
| Hypertension | 8 | (36.4) |
| Coronary artery/heart disease | 2 | (9.1) |
| Chronic/congestive heart failure | 0 | (0.0) |
| Chronic kidney disease | 0 | (0.0) |
| Cancer | 1 | (4.5) |
| COPD | 0 | (0.0) |
| Asthma | 4 | (18.2) |
| Presenting symptoms at infection, no. (%) | | |
| Fever | 16 | (72.7) |
| Cough | 17 | (77.3) |
| Anosmia/Ageusia | 13 | (59.1) |
| Pharyngitis | 8 | (36.4) |
| Headache | 14 | (63.6) |
| Confusion/Memory | 2 | (9.1) |
| Myalgias | 13 | (59.1) |
| Dyspnea | 16 | (72.7) |
| Chest pain | 8 | (36.4) |
| Nausea/Vomiting/Diarrhea | 11 | (50.0) |
| Interventions at infection, no. (%) | | |
| Steroids | 6 | (27.3) |
| Remdesivir | 0 | (0.0) |
| Tocilizumab | 1 | (4.5) |
| Long-COVID Clinic Variables | | |
| Follow up, days from infection onset, median (IQR) | 101.5 | (45.5) |
| Lingering symptoms at follow up, no. (%) | | |
| Respiratory | 16 | (72.7) |
| Cardiovascular | 6 | (27.3) |
| Neurology | 8 | (36.4) |
| Musculoskeletal | 0 | (0.0) |
| Gastro-Intestinal | 3 | (13.6) |
| Psychiatric | 1 | (4.5) |
| Cutaneous | 0 | (0.0) |
| Balance | 0 | (0.0) |
| Chest pain | 4 | (18.2) |
| Concentration | 0 | (0.0) |
| Cough | 2 | (9.1) |
| Dyspnea | 16 | (72.7) |
| Fatigue | 11 | (50.0) |
| Headache | 2 | (9.1) |
| Low mood | 1 | (4.5) |
| Anxiety | 1 | (4.5) |
| Memory | 6 | (27.3) |
| Nausea | 1 | (4.5) |
| Palpitations | 1 | (4.5) |
| Paresthesia | 1 | (4.5) |
| Smell/taste | 2 | (9.1) |
| Word finding | 1 | (4.5) |
| Non-specific | 11 | (50.0) |
| Laboratories at follow up, median (IQR) | | |
| White blood cell count | 7.1 | (1.9) |
| Neutrophils | 4.5 | (1.5) |
| Lymphocytes | 2.0 | (0.7) |
| Hemoglobin | 139.5 | (24.8) |
| Platelets | 239.5 | (64.2) |
| C-Reactive Protein (CRP) | 1.8 | (3.5) |
| Ferritin | 76.0 | (118.8) |
| Lactate Dehydrogenase (LDH) | 206.0 | (39.0) |
| Alanine Aminotransferase (ALT) | 20.0 | (11.2) |
| Interventions at follow up, no. (%) | | |
| Pulmicort | 1 | (4.5) |
| Anticoagulant | 1 | (4.5) |
| Symbicort | 10 | (45.5) |
| Ventolin | 3 | (13.6) |
| Lasix | 1 | (4.5) |
| Nasal spray | 2 | (9.1) |
| Oxygen | 2 | (9.1) |
| Physiotherapy | 4 | (18.2) |
| None | 8 | (36.4) |

TABLE 2
Acutely Ill COVID-19 Inpatient Demographics and Clinical Data

| | Ward | | ICU | |
|---|---|---|---|---|
| | Inpatients | | Inpatients | |
| Variable | (n = 22) | | (n = 22) | |
| Age (yrs), median (IQR) | 60.0 | (21.5) | 58.0 | (17.5) |
| Male sex, no. (%) | 12 | (54.5) | 12 | (54.5) |
| Weight (kg), median (IQR) | 84.8 | (14.8) | 90.0 | (28.3) |
| Height (cm), median (IQR) | 169.0 | (9.2) | 170.0 | (9.0) |
| BMI, median (IQR) | 28.6 | (5.6) | 30.5 | (7.6) |
| MODS, median (IQR) | — | | 5.0 | (1.0) |
| SOFA Score, median (IQR) | — | | 5.5 | (5.8) |
| Comorbidities, no. (%) | | | | |
| Diabetes | 4 | (18.2) | 10 | (45.5) |
| Hypertension | 9 | (40.9) | 9 | (40.9) |
| Coronary artery/heart disease | 1 | (4.5) | 2 | (9.1) |
| Chronic/congestive heart failure | 0 | (0.0) | 0 | (0.0) |
| Chronic kidney disease | 1 | (4.5) | 2 | (9.1) |
| Cancer | 3 | (13.6) | 2 | (9.1) |
| COPD | 0 | (0.0) | 1 | (4.5) |
| Presenting symptoms, no. (%) | | | | |
| Fever | 18 | (81.8) | — | |
| Cough | 18 | (81.8) | — | |
| Anosmia/Ageusia | 4 | (18.2) | — | |
| Pharyngitis | 4 | (18.2) | — | |
| Headache | 3 | (13.6) | — | |
| Myalgias | 14 | (63.6) | — | |
| Dyspnea | 20 | (90.9) | — | |
| Chest pain | 3 | (13.6) | — | |
| Nausea/Vomiting/Diarrhea | 9 | (40.9) | — | |
| Pulmonary pathology, no. (%) | | | | |
| Unilateral pneumonia | — | | 1 | (4.5) |
| Bilateral pneumonia | 21 | (95.5) | 20 | (90.9) |
| Interstitial infiltrates/R effusion | — | | 1 | (4.5) |
| Laboratories, median (IQR) | | | | |
| Hemoglobin | 129.5 | (23.0) | 118.5 | (29.8) |
| White Blood Cell count | 6.8 | (4.9) | 8.8 | (7.9) |
| Neutrophils | 5.8 | (3.9) | 7.5 | (7.4) |
| Lymphocytes | 0.8 | (0.7) | 0.7 | (0.6) |
| Platelets | 210.0 | (68.5) | 220.0 | (143.5) |
| Creatinine | 69.5 | (25.5) | 79.5 | (86.2) |
| International Normalized Ratio | 1.0 | (0.1) | 1.2 | (0.1) |
| Lactate | 1.7 | (0.9) | 1.2 | (0.8) |
| Partial thromboplastin time (PTT) | — | | 26.5 | (5.0) |
| PaO₂/FiO₂ Ratio | — | | 128.5 | (62.5) |
| Interventions, no. (%) | | | | |
| Renal replacement therapy | 0 | (0.0) | 5 | (22.7) |
| High-flow nasal cannula | 13 | (59.1) | 15 | (68.2) |
| Non-invasive mechanical ventilation | 1 | (4.5) | 6 | (27.3) |
| Invasive mechanical ventilation | 2 | (9.1) | 20 | (90.9) |
| Extracorporeal membrane | 0 | (0.0) | 1 | (4.5) |
| oxygenation | | | | |
| Tocilizumab | 2 | (9.1) | 0 | (0.0) |
| Steroids | 21 | (95.5) | 14 | (63.6) |
| Vasoactive medications | 2 | (10.0) | 18 | (81.8) |
| Antibiotics | 22 | (100.0) | 22 | (100.0) |
| Anti-virals | 4 | (18.2) | 3 | (13.6) |
| Antiplatelet | 4 | (18.2) | 17 | (77.3) |
| Anticoagulation | 22 | (100.0) | 21 | (95.5) |
| Outcomes | | | | |
| Days, median (IQR) | 9.0 | (6.8) | 15.5 | (15.0) |
| Died, no. (%) | 2 | (9.1) | 10 | (45.5) |

TABLE 3
Classification Accuracy (Random Forest) and
Area-Under-the-Curve (ROC Curve Analyses)

| | | Expression | Expression | | | |
|---|---|---|---|---|---|---|
| | | level in | level in | ROC | Bonferroni | Feature |
| | | Long- | Healthy and | Logistic | Corrected P | Importance |
| UniProt | Protein | COVID | COVID | AUC | Value | % |
| Q86WV1 | SKAP1 | 60.5 (34.4- | 0.6 (0.5- | 1 | 3.24E−10 | 1.14 |
| | | 75.6) | 0.8) | | | |
| P52564 | MAP2K6 | 68.9 (60.4- | 3.3 (1.6- | 1 | 3.24E−10 | 1.12 |
| | | 82.0) | 5.2) | | | |
| Q9H7M9 | VSIR | 42.4 (28.9- | 1.4 (1.0- | 1 | 3.24E−10 | 1.12 |
| | | 73.5) | 1.8) | | | |
| Q8TF64 | GIPC3 | 50.8 (34.5- | 3.3 (1.4- | 1 | 3.24E−10 | 1.1 |
| | | 66.6) | 7.1) | | | |
| P23560 | BDNF | 10.9 (8.4- | 0.5 (0.3- | 1 | 3.24E−10 | 1.08 |
| | | 15.3) | 0.9) | | | |
| P68106 | FKBP1B | 46.7 (28.0- | 1.2 (0.9- | 1 | 3.24E−10 | 1.06 |
| | | 67.3) | 1.5) | | | |
| P05067 | APP | 20.2 (10.5- | 1.4 (1.0- | 1 | 3.24E−10 | 1.06 |
| | | 25.3) | 1.7) | | | |
| P19876 | CXCL3 | 56.5 (38.2- | 1.5 (0.7- | 1 | 3.24E−10 | 1.04 |
| | | 67.5) | 3.0) | | | |
| Q92765 | FRZB | 0.2 (0.2- | 1.5 (1.1- | 1 | 3.24E−10 | 1.02 |
| | | 0.3) | 2.0) | | | |
| P42830 | CXCL5 | 65.3 (34.2- | 0.8 (0.5- | 1 | 3.24E−10 | 1 |
| | | 98.4) | 1.1) | | | |
| Q9HCN6 | GP6 | 36.7 (25.7- | 1.5 (0.9- | 1 | 3.24E−10 | 0.96 |
| | | 45.7) | 2.5) | | | |
| P20340 | RAB6A | 22.7 (8.0- | 0.3 (0.3- | 1 | 3.24E−10 | 0.96 |
| | | 42.6) | 0.5) | | | |
| P09960 | LTA4H | 3.2 (2.8- | 0.0 (0.0- | 1 | 3.24E−10 | 0.96 |
| | | 3.9) | 0.1) | | | |
| P02775 | PPBP | 16.7 (10.7- | 0.5 (0.2- | 1 | 3.24E−10 | 0.94 |
| | | 19.8) | 1.1) | | | |
| Q96A25 | TMEM106A | 11.9 (9.0- | 1.6 (1.0- | 1 | 3.24E−10 | 0.94 |
| | | 23.8) | 2.0) | | | |
| P16109 | SELP | 8.7 (6.4- | 1.1 (0.8- | 1 | 3.24E−10 | 0.94 |
| | | 11.9) | 1.4) | | | |
| P02751 | FN1 | 0.2 (0.2- | 1.3 (1.0- | 1 | 3.24E−10 | 0.92 |
| | | 0.2) | 1.6) | | | |
| P12532 | CKMT1A_{—} | 0.1 (0.0- | 2.4 (1.8- | 1 | 3.24E−10 | 0.92 |
| | CKMT1B | 0.1) | 3.1) | | | |
| Q86YW5 | TREML1 | 15.7 (10.8- | 1.1 (0.8- | 1 | 3.24E−10 | 0.9 |
| | | 21.1) | 1.6) | | | |
| O60243 | HS6ST1 | 0.2 (0.1- | 1.4 (0.8- | 1 | 3.24E−10 | 0.9 |
| | | 0.2) | 2.7) | | | |
| Q9HD42 | CHMP1A | 7.7 (5.9- | 1.7 (0.7- | 1 | 3.24E−10 | 0.88 |
| | | 8.7) | 2.5) | | | |
| P29965 | CD40LG | 20.8 (10.1- | 1.1 (0.7- | 1 | 3.24E−10 | 0.88 |
| | | 25.3) | 1.8) | | | |
| O14944 | EREG | 40.8 (19.0- | 0.7 (0.5- | 1 | 3.24E−10 | 0.88 |
| | | 61.1) | 1.1) | | | |
| Q9Y2X7 | GIT1 | 23.0 (15.1- | 3.6 (1.7- | 1 | 3.24E−10 | 0.86 |
| | | 32.4) | 5.2) | | | |
| P42575 | CASP2 | 32.5 (18.6- | 2.8 (1.8- | 1 | 3.24E−10 | 0.86 |
| | | 53.1) | 4.0) | | | |
| P01133 | EGF | 53.1 (31.3- | 1.2 (0.6- | 1 | 3.24E−10 | 0.84 |
| | | 65.4) | 2.4) | | | |
| Q9H0P0 | NT5C3A | 80.7 (70.5- | 4.8 (2.0- | 1 | 3.24E−10 | 0.82 |
| | | 113.6) | 9.3) | | | |
| P22004 | BMP6 | 0.2 (0.1- | 1.3 (0.9- | 1 | 3.24E−10 | 0.76 |
| | | 0.3) | 1.4) | | | |
| P40197 | GP5 | 2.2 (2.0- | 0.6 (0.4- | 1 | 3.24E−10 | 0.74 |
| | | 2.4) | 0.9) | | | |
| P12931 | SRC | 72.0 (53.7- | 4.2 (2.0- | 1 | 3.24E−10 | 0.72 |
| | | 109.6) | 7.4) | | | |
| Q07108 | CD69 | 84.8 (59.9- | 5.2 (2.4- | 1 | 3.24E−10 | 0.7 |
| | | 120.9) | 9.0) | | | |
| Q9UBW5 | BIN2 | 100.2 (52.2- | 3.8 (2.3- | 1 | 3.24E−10 | 0.66 |
| | | 203.9) | 6.5) | | | |
| P01024 | C3 | 1.5 (1.2- | 0.2 (0.1- | 1 | 3.24E−10 | 0.62 |
| | | 1.9) | 0.2) | | | |
| Q9Y6A5 | TACC3 | 188.9 (131.0- | 6.0 (2.8- | 1 | 3.47E−10 | 1.16 |
| | | 306.6) | 11.1) | | | |
| Q9ULL4 | PLXNB3 | 6.5 (4.6- | 1.0 (0.8- | 1 | 3.47E−10 | 1.08 |
| | | 9.9) | 1.4) | | | |
| Q9Y2Y0 | ARL2BP | 15.3 (11.5- | 1.3 (0.9- | 1 | 3.47E−10 | 1 |
| | | 29.9) | 1.7) | | | |
| Q9UHD8 | SEPTIN9 | 32.2 (24.4- | 1.8 (1.3- | 1 | 3.47E−10 | 0.86 |
| | | 66.8) | 2.4) | | | |
| Q9NUY8 | TBC1D23 | 54.9 (36.9- | 3.5 (1.9- | 1 | 3.47E−10 | 0.82 |
| | | 82.7) | 6.4) | | | |
| O75351 | VPS4B | 17.2 (11.9- | 1.8 (1.0- | 1 | 3.47E−10 | 0.76 |
| | | 25.6) | 2.5) | | | |
| P40818 | USP8 | 12.6 (6.6- | 0.5 (0.4- | 1 | 3.47E−10 | 0.74 |
| | | 21.7) | 0.9) | | | |
| P55957 | BID | 6.6 (4.5- | 0.4 (0.2- | 1 | 3.72E−10 | 1.22 |
| | | 10.1) | 0.6) | | | |
| Q9UJU6 | DBNL | 68.4 (50.7- | 4.2 (1.7- | 1 | 3.72E−10 | 1.1 |
| | | 98.4) | 6.6) | | | |
| Q99616 | CCL13 | 23.0 (20.1- | 2.2 (1.6- | 1 | 3.72E−10 | 1.06 |
| | | 30.9) | 3.2) | | | |
| Q9UKW4 | VAV3 | 241.4 (101.6- | 4.4 (2.1- | 1 | 3.72E−10 | 0.98 |
| | | 622.6) | 9.2) | | | |
| Q5VY43 | PEAR1 | 2.2 (2.0- | 1.0 (0.9- | 1 | 3.72E−10 | 0.98 |
| | | 2.8) | 1.2) | | | |
| P13501 | CCL5 | 12.3 (8.6- | 1.1 (0.3- | 1 | 3.72E−10 | 0.96 |
| | | 19.4) | 1.8) | | | |
| Q14790 | CASP8 | 26.4 (17.7- | 0.6 (0.3- | 1 | 3.72E−10 | 0.94 |
| | | 36.5) | 0.9) | | | |
| O00194 | RAB27B | 47.2 (41.1- | 12.0 (3.9- | 1 | 3.72E−10 | 0.9 |
| | | 60.8) | 23.0) | | | |
| Q13976 | PRKG1 | 199.8 (123.7- | 8.8 (3.5- | 1 | 3.72E−10 | 0.88 |
| | | 557.2) | 17.3) | | | |
| Q9UIB8 | CD84 | 3.5 (3.0- | 1.2 (0.9- | 1 | 3.72E−10 | 0.86 |
| | | 3.8) | 1.5) | | | |
| Q9BQS7 | HEPH | 1.1 (0.9- | 0.4 (0.3- | 1 | 3.72E−10 | 0.86 |
| | | 1.1) | 0.4) | | | |
| Q6P589 | TNFAIP8L2 | 14.3 (7.8- | 0.9 (0.7- | 1 | 3.72E−10 | 0.82 |
| | | 20.0) | 1.3) | | | |
| Q8TE58 | ADAMTS15 | 1.2 (0.9- | 5.6 (4.1- | 1 | 3.72E−10 | 0.78 |
| | | 1.7) | 8.0) | | | |
| P55273 | CDKN2D | 32.7 (23.9- | 1.8(1.0- | 1 | 3.72E−10 | 0.74 |
| | | 37.6) | 3.1) | | | |
| Q15389 | ANGPT1 | 14.3 (10.0- | 1.1 (0.5- | 1 | 3.72E−10 | 0.72 |
| | | 16.8) | 1.9) | | | |
| O75167 | PHACTR2 | 23.9 (14.4- | 2.0 (1.1- | 1 | 3.72E−10 | 0.6 |
| | | 38.0) | 3.2) | | | |
| Q9UNE0 | EDAR | 17.7 (12.4- | 1.1 (0.8- | 1 | 3.98E−10 | 1.34 |
| | | 30.2) | 1.5) | | | |
| O95644 | NFATC1 | 19.2 (9.3- | 1.6 (1.0- | 1 | 3.98E−10 | 1.06 |
| | | 22.6) | 2.3) | | | |
| Q13561 | DCTN2 | 111.7 (59.5- | 3.5 (2.1- | 1 | 3.98E−10 | 0.88 |
| | | 196.2) | 6.3) | | | |
| Q92609 | TBC1D5 | 80.6 (57.7- | 6.7 (3.3- | 1 | 3.98E−10 | 0.86 |
| | | 111.7) | 12.6) | | | |
| Q16206 | ENOX2 | 5.9 (3.4- | 1.3 (1.0- | 1 | 3.98E−10 | 0.8 |
| | | 12.0) | 1.6) | | | |
| Q08AG7 | MZT1 | 16.2 (10.8- | 1.6 (1.3- | 1 | 3.98E−10 | 0.7 |
| | | 39.5) | 2.2) | | | |
| Q12765 | SCRN1 | 25.7 (18.8- | 1.5 (1.0- | 1 | 4.27E−10 | 1.1 |
| | | 35.4) | 2.5) | | | |
| P04085 | PDGFA | 11.4 (8.1- | 0.7(0.3- | 0.99 | 4.27E−10 | 0.96 |
| | | 13.1) | 1.2) | | | |
| P23743 | DGKA | 43.8 (26.5- | 2.8 (1.9- | 1 | 4.27E−10 | 0.94 |
| | | 81.0) | 4.5) | | | |
| Q15762 | CD226 | 6.6 (4.6- | 0.9 (0.7- | 1 | 4.27E−10 | 0.92 |
| | | 8.4) | 1.5) | | | |
| P53990 | IST1 | 18.6 (14.0- | 1.7 (0.8- | 1 | 4.27E−10 | 0.88 |
| | | 26.0) | 2.7) | | | |
| Q13576 | IQGAP2 | 67.0 (47.0- | 2.3 (1.3- | 1 | 4.27E−10 | 0.86 |
| | | 105.1) | 4.1) | | | |
| O60496 | DOK2 | 39.8 (20.9- | 3.5 (1.7- | 1 | 4.57E−10 | 1.12 |
| | | 57.3) | 5.3) | | | |
| Q92783 | STAM | 11.5 (8.7- | 1.5 (1.0- | 1 | 4.57E−10 | 1.12 |
| | | 18.3) | 1.9) | | | |
| Q9NR12 | PDLIM7 | 120.1 (104.7- | 4.2 (2.0- | 1 | 4.57E−10 | 0.96 |
| | | 160.9) | 9.7) | | | |
| P55039 | DRG2 | 9.3 (4.6- | 1.2 (0.9- | 1 | 4.57E−10 | 0.88 |
| | | 12.8) | 1.7) | | | |
| Q6ZRY4 | RBPMS2 | 167.1 (141.3- | 15.5 (4.1- | 1 | 4.57E−10 | 0.86 |
| | | 190.4) | 33.5) | | | |
| Q9BX10 | GTPBP2 | 55.3 (36.0- | 2.6 (1.5- | 0.99 | 4.57E−10 | 0.8 |
| | | 122.8) | 4.3) | | | |
| P80162 | CXCL6 | 9.2 (7.9- | 1.1 (0.8- | 1 | 4.57E−10 | 0.74 |
| | | 11.7) | 1.6) | | | |
| Q9UKU9 | ANGPTL2 | 0.3 (0.2- | 1.4 (1.1- | 0.99 | 4.89E−10 | 1.04 |
| | | 0.5) | 1.9) | | | |
| P42574 | CASP3 | 23.3 (18.1- | 4.5 (1.8- | 0.99 | 4.89E−10 | 0.96 |
| | | 44.7) | 7.2) | | | |
| Q8NEZ2 | VPS37A | 9.9 (6.8- | 1.5 (1.0- | 1 | 4.89E−10 | 0.96 |
| | | 13.2) | 2.0) | | | |
| P49137 | MAPKAPK2 | 13.0 (9.6. | 2.0 (1.4- | 0.99 | 4.89E−10 | 0.9 |
| | | 20.7) | 3.3) | | | |
| O60884 | DNAJA2 | 35.3 (27.5- | 4.3 (1.6- | 1 | 4.89E−10 | 0.9 |
| | | 45.2) | 6.4) | | | |
| Q15276 | RABEP1 | 24.6 (18.5- | 2.6 (1.7- | 1 | 4.89E−10 | 0.84 |
| | | 62.9) | 4.4) | | | |
| P13807 | GYS1 | 16.2 (7.9- | 0.6 (0.4- | 1 | 4.89E−10 | 0.62 |
| | | 23.2) | 1.2) | | | |
| Q8N1Q1 | CA13 | 62.1 (38.6- | 3.1 (1.5- | 0.99 | 5.23E−10 | 0.8 |
| | | 96.7) | 4.5) | | | |
| Q5SW79 | CEP170 | 48.5 (33.9- | 7.5 (2.6- | 1 | 5.24E−10 | 0.98 |
| | | 54.8) | 10.9) | | | |
| P09104 | ENO2 | 8.4 (4.9- | 1.6 (1.1- | 0.99 | 5.24E−10 | 0.46 |
| | | 16.1) | 2.5) | | | |
| Q8IU57 | IFNLR1 | 0.3 (0.3- | 1.4 (1.0- | 0.99 | 5.24E−10 | 0.36 |
| | | 0.4) | 1.9) | | | |
| O43665 | RGS10 | 6.3 (4.1- | 1.5 (1.3- | 1 | 5.60E−10 | 1.26 |
| | | 8.5) | 1.7) | | | |
| P61244 | MAX | 18.2 (7.7- | 1.1 (0.7- | 1 | 5.60E−10 | 1.02 |
| | | 34.2) | 1.7) | | | |
| O75190 | DNAJB6 | 47.4 (35.1- | 3.3 (1.9- | 1 | 5.60E−10 | 0.98 |
| | | 88.9) | 5.3) | | | |
| P59780 | AP3S2 | 8.4 (5.6- | 1.2 (0.8- | 1 | 5.60E−10 | 0.9 |
| | | 11.2) | 1.5) | | | |
| Q9NWM8 | FKBP14 | 14.0 (6.9- | 1.4 (0.9- | 1 | 5.60E−10 | 0.74 |
| | | 33.7) | 1.8) | | | |
| Q96RT1 | ERBIN | 21.5 (14.0- | 2.2 (1.3- | 1 | 5.60E−10 | 0.68 |
| | | 31.2) | 3.5) | | | |
| O00161 | SNAP23 | 12.0 (7.3- | 0.8 (0.4- | 1 | 6.00E−10 | 0.98 |
| | | 14.6) | 1.5) | | | |
| O94986 | CEP152 | 13.3 (8.2- | 1.6 (0.9- | 1 | 6.00E−10 | 0.8 |
| | | 35.2) | 2.3) | | | |
| Q9BV40 | VAMP8 | 36.2 (26.4- | 5.5 (2.4- | 1 | 6.42E−10 | 0.58 |
| | | 46.7) | 8.2) | | | |
| P51671 | CCL11 | 2.8 (2.5- | 1.1 (0.8- | 1 | 6.87E−10 | 0.94 |
| | | 3.0) | 1.4) | | | |
| P78352 | DLG4 | 39.5 (23.6- | 2.3 (1.4- | 0.99 | 6.87E−10 | 0.84 |
| | | 122.8) | 4.2) | | | |
| Q96IU4 | ABHD14B | 15.6 (11.3- | 0.6 (0.4- | 1 | 6.87E−10 | 0.7 |
| | | 20.3) | 0.8) | | | |
| P05154 | SERPINA5 | 1.6 (1.1- | 0.4 (0.3- | 0.99 | 6.87E−10 | 0.46 |
| | | 1.7) | 0.6) | | | |
| P02776 | PF4 | 8.7 (6.1- | 0.7 (0.5- | 0.98 | 8.41E−10 | 0.54 |
| | | 9.7) | 1.3) | | | |
| Q9UDT6 | CLIP2 | 47.9 (32.5- | 7.1 (3.0- | 0.98 | 9.00E−10 | 0.92 |
| | | 65.9) | 13.2) | | | |
| Q92583 | CCL17 | 8.5 (5.5- | 0.6 (0.4- | 0.99 | 9.62E−10 | 0.86 |
| | | 12.2) | 1.2) | | | |
| Q9P2T1 | GMPR2 | 30.9 (16.4- | 3.4 (1.9- | 1 | 9.62E−10 | 0.8 |
| | | 58.2) | 5.3) | | | |
| P02745 | C1QA | 0.7 (0.6- | 1.6 (1.2- | 0.98 | 9.62E−10 | 0.22 |
| | | 0.8) | 2.0) | | | |
| Q9HD26 | GOPC | 15.5 (9.9- | 3.1 (1.7- | 1 | 1.18E−09 | 0.64 |
| | | 24.1) | 4.5) | | | |
| Q9Y258 | CCL26 | 13.6 (9.6- | 0.9 (0.6- | 0.98 | 1.26E−09 | 0.92 |
| | | 20.7) | 1.4) | | | |
| P41227 | NAA10 | 30.9 (17.2- | 4.1 (1.8- | 1 | 1.35E−09 | 0.42 |
| | | 36.9) | 6.0) | | | |
| Q8N129 | CNPY4 | 9.5 (5.9- | 1.4 (1.0- | 1 | 1.54E−09 | 0.56 |
| | | 16.5) | 2.2) | | | |
| P30405 | PPIF | 3.9 (2.6- | 1.3 (1.1- | 0.97 | 1.54E−09 | 0.28 |
| | | 14.7) | 1.5) | | | |
| Q9UNK0 | STX8 | 18.1 (12.7- | 2.0 (1.1- | 0.99 | 1.64E−09 | 0.72 |
| | | 21.6) | 3.3) | | | |
| Q8NBI3 | DRAXIN | 0.4 (0.3- | 1.6 (1.1- | 0.99 | 1.88E−09 | 0.32 |
| | | 0.6) | 2.3) | | | |
| Q6UY14 | ADAMTSL4 | 0.8 (0.6- | 4.4 (1.9- | 0.99 | 1.88E−09 | 0.1 |
| | | 1.0) | 7.7) | | | |
| P31431 | SDC4 | 3.3 (2.6- | 0.3 (0.1- | 0.99 | 2.01E−09 | 0.64 |
| | | 4.3) | 0.5) | | | |
| Q9NRY6 | PLSCR3 | 21.7 (16.1- | 3.4 (2.2- | 0.99 | 2.14E−09 | 0.24 |
| | | 31.8) | 4.7) | | | |
| Q9BSW2 | CRACR2A | 56.2 (41.0- | 6.1 (2.6- | 0.97 | 2.29E−09 | 0.92 |
| | | 143.9) | 9.9) | | | |
| Q99683 | MAP3K5 | 72.3 (38.9- | 5.9 (3.0- | 0.96 | 3.87E−09 | 0.9 |
| | | 115.7) | 11.1) | | | |
| P09341 | CXCL1 | 24.9 (18.3- | 2.3 (1.2- | 0.97 | 4.13E−09 | 0.1 |
| | | 34.1) | 4.0) | | | |
| Q15797 | SMAD1 | 4.3 (2.8- | 1.3 (0.9- | 0.97 | 5.02E−09 | 1.16 |
| | | 7.9) | 1.6) | | | |
| Q6UWW8 | CES3 | 0.3 (0.2- | 0.1 (0.1- | 0.91 | 7.19E−07 | 0.06 |
| | | 0.5) | 0.1) | | | |

Note:
Long-COVID, Long-COVID outpatients (n = 22); Healthy and COVID, health control subjects (n = 22), acutely Ward COVID inpatients (n = 22), acutely ill ICU COVID inpatients (n = 22). Mann-Whitney U test with Bonferroni multiple comparisons correction.

TABLE 4
Expression of the Top 9 Proteins in Specific Groups

| | | Expression | Expression | Expression | Expression | | |
|---|---|---|---|---|---|---|---|
| | | level in | level | level in | level in | | |
| UniProt | Protein | Long-COVID | in ICU | Ward | Healthy | P Value | Change |
| P42830 | CXCL5 | 65.3 (34.2- | 0.9 (0.5- | 0.9 (0.6- | 0.6 (0.3- | <0.00001 | ↑ |
| | | 98.4) | 1.2) | 1.4) | 0.8) | | |
| P59780 | AP3S2 | 8.4 (5.6- | 1.4 (1.2- | 1.4 (1.2- | 0.7(0.7- | <0.00001 | ↑ |
| | | 11.2) | 2.0) | 1.5) | 0.8) | | |
| P61244 | MAX | 18.2 (7.7- | 1.4 (0.8- | 1.4 (0.8- | 0.8 (0.6- | <0.00001 | ↑ |
| | | 34.2) | 2.0) | 1.9) | 1.1) | | |
| Q9NR12 | PDLIM7 | 120.1 (104.7- | 5.8 (3.7- | 8.7 (5.2- | 1.2 (0.8- | <0.00001 | ↑ |
| | | 160.9) | 18.1) | 11.6) | 2.1) | | |
| Q9UNE0 | EDAR | 17.7 (12.4- | 1.5 (0.9- | 0.9 (0.7- | 1.1 (0.9- | <0.00001 | ↑ |
| | | 30.2) | 2.2) | 1.2) | 1.3) | | |
| P09960 | LTA4H | 3.2 (2.8- | 0.1 (0.1- | 0.1 (0.0- | 0.0 (0.0- | <0.00001 | ↑ |
| | | 3.9) | 0.1) | 0.1) | 0.0) | | |
| Q9BSW2 | CRACR2A | 56.2 (41.0- | 8.2 (6.3- | 9.1 (6.2- | 1.9 (1.2- | <0.00001 | ↑ |
| | | 143.9) | 11.7) | 12.7) | 2.5) | | |
| P19876 | CXCL3 | 56.5 (38.2- | 2.4 (1.5- | 2.7 (1.5- | 0.6 (0.3- | <0.00001 | ↑ |
| | | 67.5) | 3.1) | 4.2) | 0.8) | | |
| Q92765 | FRZB | 0.2 (0.2- | 1.9 (1.5- | 1.8 (1.5- | 1.2 (1.0- | <0.00001 | ↓ |
| | | 0.3) | 2.1) | 2.0) | 1.3) | | |

Note:
P Value (Kruskal-Wallis) compares Healthy vs Ward vs ICU vs Long-COVID. Change represents the change Long-COVID relative to acute COVID and healthy controls.

TABLE 5
Expression NLP Categories by Organ System of the Top 119 Proteins

| Organ System | Proteins | Keywords |
|---|---|---|
| Cardiovascular | FN1, BDNF, MAP2K6, APP, | abdominal aorta, aorta, aorta |
| | FKBP1B, FRZB, CASP2, | extracellular, aortic, aortic intima, aortic |
| | TBC1D23, PEAR1, DRG2, | valves, arteries, arteriolar tree, artery, |
| | VPS37A, CASP3, ANGPTL2, | ascending aorta, atria, atrium, atrium |
| | DNAJB6, ERBIN, MAX, | cardiomyocytes, blood, blood vessel |
| | SERPINA5, ABHD14B, GMPR2, | walls, blood vessels, capillaries, capillary |
| | CCL26, STX8, ADAMTSL4, | endothelium, cardiac, cardiac, cardiac |
| | PLSCR3, MAP3K5, SMAD1 | atria, cardiac muscle, cardiac muscles, |
| | | cardiovascular, blood vessels, coronary, |
| | | coronary arteries, coronary artery, |
| | | coronary artery smooth muscle, dermal |
| | | blood vessels, ductus arteriosus, fetal |
| | | heart, heart, heart muscle, heart muscle, |
| | | heart spleen, heart ventricle, heart |
| | | ventricles, hearts, inter-ventricular |
| | | septum, large arteries, large vessels, |
| | | lateral ventricle, mammary artery, |
| | | myocardium, myocardium, periosteum, |
| | | right atrium, right ventricle, skin blood |
| | | vessels, small capillaries, small vessel |
| | | endothelium, small vessels, stromal |
| | | vascular, system vessels, thoracic aorta, |
| | | umbilical cord artery, vasa vasorum, |
| | | vascular, vascular, vascular capillary |
| | | network, vascular endothelium, vascular |
| | | smooth muscle, vascular structure, |
| | | vascular structures, vascular system, |
| | | vascular-rich organs, vasculature, |
| | | vasculature, vein, veins, ventricle, |
| | | ventricles, ventricular, ventricular |
| | | trabeculae, vessel wall, vessels |
| Digestive | GIPC3, CASP2, BIN2, SRC, FRZB, | adult colon epithelium, adult pancreas, |
| | EREG, APP, MAP2K6, EGF, | adult stomach, appendix, appendix, bile |
| | NT5C3A, TBC1D23, PLXNB3, | ducts, bowel, buccal mucosa, cecum, |
| | CCL13, ADAMTS15, BID, CASP8, | colon, colon, colon, colon intestine, colon |
| | PEAR1, DRG2, PDLIM7, DOK2, | mucosa, colon-rectum muscularis |
| | VPS37A, ANGPTL2, CASP3, | mucosae epithelium, colonic epithelium, |
| | CA13, DNAJB6, ERBIN, MAX, | colorectal, deodenum, descending colon, |
| | SERPINA5, ABHD14B, CCL17, | digestive, digestive system epithelium, |
| | GMPR2, STX8, ADAMTSL4, | duodenum, duodenum, duodenum |
| | PLSCR3, MAP3K5, CES3 | mucosal crypts, esophagus, esophagus, |
| | | exocrine pancreas, exocrine pancreatic |
| | | ducts, fetal colon, fetal liver, fetal liver, |
| | | fetal liver, fundic epithelium, gall |
| | | bladder, gallbladder, gallbladder bile, |
| | | gastric, gastric antrum, gastric mucosa, |
| | | gastrointestinal, gastrointestinal, |
| | | gastrointestinal epithelia, |
| | | gastrointestinal epithelium, |
| | | gastrointestinal tract, gut, hepatic, |
| | | hepatic endothelia, ileocecum, ileum, |
| | | ileum, intestinal, intestinal, intestinal |
| | | brush border, intestinal crypts, intestinal |
| | | epithelia, intestinal epithelium, intestinal |
| | | tract, intestine, intestine, intestines, |
| | | islets, jejunum, jejunum, jejunum brush |
| | | border, langerhans, large intestine, large |
| | | intestines, liver, liver, liver skeletal |
| | | muscle, mouth, mucous acini, non- |
| | | cancerous liver, normal stomach, |
| | | omentum, oral cavity, oral epithelia, oral |
| | | epithelium, oral tongue, palatal epithelia, |
| | | palatal shelf, palate, pancreas, pancreas |
| | | acinar ductal epithelium, pancreas islets, |
| | | pancreatic, pancreatic acini, pancreatic |
| | | beta-cells, pancreatic duct, pancreatic |
| | | islets, parotid, parotid, parotid gland, |
| | | parotid saliva, parotid salivary gland |
| | | intralobular ducts, rectal, rectum, |
| | | rectum, salivary, salivary gland, salivary |
| | | gland, salivary glands, small intestine, |
| | | small intestine, small intestine, small |
| | | intestine, small intestines, stomach, |
| | | stomach wall, sublingual gland, |
| | | sublingual glands, submandibular, |
| | | submandibular gland, submaxillary |
| | | glands, teeth, tongue, tooth, transverse, |
| | | transverse colon |
| Endocrine | VSIR, BDNF, CHMP1A, FRZB, | adrenal, adrenal, adrenal cortex, adrenal |
| | BIN2, EREG, CASP2, PLXNB3, | gland, adrenal gland, adrenal glands, |
| | PRKG1, PEAR1, VAV3, BID, | adrenal glands, adrenal glomerulosa, |
| | NFATC1, VPS37A, ANGPTL2, | adrenals, adult placenta, ducts, |
| | DNAJB6, SNAP23, ABHD14B, | endocrine glands, fetal adrenal, fetal |
| | GMPR2, STX8, ADAMTSL4, | placenta, gland, glands, hypophysis, |
| | PLSCR3 | hypothalamus, hypothalamus, intestinal |
| | | glands, lacrimal gland, lateral |
| | | hypothalamus, parathyroid, parathyroid |
| | | gland, phaeochromocytoma, pineal |
| | | gland, pituitary, pituitary gland, pituitary |
| | | gland, placenta, placenta, placenta, |
| | | placenta syncytiotrophoblasts, placenta |
| | | vascular, placenta vascular endothelium, |
| | | placental, placental, placental |
| | | endothelium, placental membranes, |
| | | placental stem villi vessels, placental |
| | | vascular, placental villi, placental villi, |
| | | placentas, steroidogenic glands, |
| | | submucosal gland, submucosal glands, |
| | | term placenta, thyroid, thyroid, thyroid |
| | | follicles, thyroid gland, thyroid gland, |
| | | thyroid glands |
| Integumentary | CCL5, EDAR, SERPINA5, | anagen follicles, basal epidermal layer, |
| | ADAMTSL4 | basal layer, basal skin layer, club hair, |
| | | dermal papilla, dermis, eccrine sweat, |
| | | eccrine sweat glands, exocrine, fetal |
| | | follicles, fetal skin, follicular, follicular |
| | | fluid, hair, hair fibers, hair follicle, hair |
| | | follicles, nail bed epithelium, nail matrix, |
| | | palmoplantar epidermis, scalp, scalp |
| | | follicles, scalp skin, scar lesional skin, |
| | | sebaceous gland, skin, skin epidermis, |
| | | stratum corneum, stratum granulosum, |
| | | stratum spinosum, sweat, sweat ducts, |
| | | sweat gland, sweat gland ducts, sweat |
| | | glands, sweat glands, upper spinous |
| | | layers |
| Lymphatic | VSIR, GIPC3, APP, NT5C3A, | -rich red pulp, adenoid, adult lymph |
| | FKBP1B, SRC, BIN2, TBC1D23, | nodes, beta-cells, bone marrow, fetal |
| | SEPTIN9, CD84, VAV3, BID, | spleen, fetal spleen, fetal thymus, fetal |
| | CASP8, CCL13, NFATC1, DRG2, | thymus, fetal thymus, fetal tonsils, |
| | DOK2, GTPBP2, PDLIM7, | germinal center, germinal centers, |
| | ANGPTL2, CASP3, CA13, | hematopoietic, hematopoietic, immune |
| | DNAJB6, ERBIN, SERPINA5, | system, lymph, lymph, lymph node, |
| | ABHD14B, CCL17, GMPR2, | lymph node-containing, lymph nodes, |
| | STX8, ADAMTSL4, PLSCR3 | lymphatic, lymphatic vessels, lymphatics, |
| | | lymphocytic compartment, lymphoid, |
| | | lymphoid node, lymphoid organs, |
| | | lymphoid organs, mantle zones, |
| | | mesenteric lymph nodes, peripheral |
| | | lymph nodes, peyer patches, peyer's |
| | | patches, peyers's patches, red pulp, |
| | | secondary lymphoid, spleen, spleen, |
| | | thymic medulla, thymus, thymus, thymus |
| | | epithelium, thymus medulla, tonsil, |
| | | tonsil, tonsils |
| Musculoskeletal | BDNF, MAP2K6, FRZB, CASP2, | adult skeletal muscle, appendicular |
| | TBC1D23, SEPTIN9, VAV3, | skeleton, articular, articular cartilage, |
| | CASP8, PEAR1, NFATC1, | articular cartilages, articular hyaline |
| | PDLIM7, DRG2, VPS37A, | cartilage, bone, bone matrix, bone- |
| | ANGPTL2, CASP3, ENO2, | forming sites, bone-forming surfaces, |
| | DNAJB6, ERBIN, MAX, | bones, bones, calvaria, calvaria, carpal |
| | SERPINA5, ABHD14B, GMPR2, | bones, cartilage, cartilages, cartilaginous, |
| | STX8, ADAMTSL4, PLSCR3, | cartilaginous cores, cortical plate, cranial |
| | SMAD1 | cartilage, deep zone cartilage, dental |
| | | enamel, dental papilla, dental pulp, |
| | | dentin, epiphysis, fetal cartilage, fetal |
| | | perichondrium, ganglia, hip articular |
| | | cartilage, hypertrophic cartilage, |
| | | intervertebral disk, invertebral disk, joint |
| | | capsule, joint cartilage, joints, ligament, |
| | | ligaments, long bone, long bones, lumbar |
| | | disk, metaphyseal bone, muscle, muscle, |
| | | muscle fibers, muscles, osseous, |
| | | periodontium, rib bone, sarcomeric |
| | | muscle, skeletal, skeletal muscle, skeletal |
| | | muscle, skeletal muscle), skeletal |
| | | muscles, skeletal muscles, spinal |
| | | muscular, striated muscle, striated |
| | | muscle, striated muscles, synaptic fibers, |
| | | synovial, synovial fluid, synovium, tarsal |
| | | bones, tendon, trabecular bone, |
| | | vertebrae |
| Nervous | FN1, BDNF, GIPC3, HS6ST1, | adrenal medulla, adult cns, adult nervous |
| | APP, FKBP1B, FRZB, SRC, | central system, amygdala, anterior horn, |
| | CASP2, TBC1D23, SEPTIN9, | auerbach plexus, axons, basal ganglia, |
| | PLXNB3, PEAR1, VAV3, CASP8, | blood-brain, brain, brain, brain, brain |
| | NFATC1, GTPBP2, VPS37A, | cortex, brain neocortex, brain regions, |
| | CASP3, DNAJB6, ERBIN, MAX, | brain stem, brain structures, brainstem, |
| | LG4, GMPR2, STX8, ADAMTSL4 | brainstem, bruch's membrane, caudate |
| | | nuclei, caudate nucleus, caudate region, |
| | | central, central nervous, central nervous |
| | | system, cerebellar nuclei, cerebellum, |
| | | cerebellum, cerebral cortex, cerebral |
| | | spinal, cerebro-spinal fluid, |
| | | choriocapillaris, choroid, choroid plexus, |
| | | ciliary body, ciliary border, ciliary |
| | | nonpigmented epithelium, circumvallate |
| | | papillae, cns, cochlea, cochlea, cone |
| | | photoreceptors, conjunctival epithelia, |
| | | conjunctival epithelium, cornea, cornea, |
| | | corneal, corneal epithelium, corneal |
| | | stromal layer, corpus callosum, corpus |
| | | luteum, corpus region, cortex, cortical |
| | | layers, cranial ganglia, dentate gyrus, |
| | | dentate nucleus, diencephalon, dorsal |
| | | root ganglia, dorsal root ganglia, dorsal |
| | | root ganglion, drg, embryonic retina, |
| | | extraocular smooth muscle, eye, eye |
| | | anterior segment, eye lens, eyes, fetal |
| | | brain, fetal brain, fetal brain, fetal brains, |
| | | fetal cerebellum, fetal eye, fetal frontal |
| | | lobe, fetal retinal pigment epithelium, |
| | | fetal substantia nigra, frontal cortex, |
| | | frontal lobe, fusiform gyrus, ganglion cell |
| | | layer, germinal neuroepithelium, globus |
| | | pallidus, hippocampal ca1, hippocampal |
| | | dentate gyrus, hippocampal subfields, |
| | | hippocampus, hippocampus, inner ear, |
| | | insula, iris, lumbar, medulla, medulla, |
| | | medulla oblongata, medulla region, |
| | | midbrain structures, motor cortices, |
| | | myelinated structures, neocortex, |
| | | neocortical regions, nerve fiber layer, |
| | | nervous, nervous system, neural, neural |
| | | retina, neuroendocrine, neuron, |
| | | neuronal, neuroretina, neutrophils |
| | | thyroid gland, nucleus accumbens, |
| | | occipital, occipital lobe, occipital pole, |
| | | olfactory bulb, olfactory epithelium, |
| | | olfactory lobe, olfactory tubercles, |
| | | ophthalmic nerve, optic nerve, papillary |
| | | sphincter, parahippocampal cortex, |
| | | paraolfactory gyri, parietal lobe, parietal |
| | | lobes, periaxonal myelin, peripheral |
| | | nerve, peripheral nervous system, |
| | | peripheral nervous systems, peripheral |
| | | retina, photoreceptor outer, pigmented |
| | | epithelium, pns neuroectoderm, pons, |
| | | pons, posterior perisylvian, postrema, |
| | | postsynaptic structures, prefrontal |
| | | cortex, putamen, putamen, retina, |
| | | retina, retina pigment epithelium, |
| | | retinal, retinal cone photoreceptors, |
| | | retinal pericytes, retinal pigment |
| | | epithelia, retinal pigment epithelium, |
| | | retinal rod, rod, rod photoreceptors, |
| | | rolandic area, rostral segment, sclera, |
| | | spinal chord, spinal chord, spinal cord, |
| | | spinal cord, spinal cord, spinal cordon, |
| | | stria vascularis, subiculum, substantia |
| | | nigra, subthalamic nucleus, sustantia |
| | | nigra, sympathetic, synaptic fibers, |
| | | telencephalon, temporal cortex, |
| | | temporal gyrus, temporal lobe, temporal |
| | | lobes, thalamus, thalamus, ventral |
| | | striatum, vertebrae, vestibular system, |
| | | vestibule |
| Reproductive | VSIR, BDNF, EGF, SKAP1, | adult testis, bartholin's, breast, breast, |
| | TBC1D23, SEPTIN9, RAB27B, | breast cyst, cerebrum, cervical, cervical |
| | CASP8, PEAR1, IQGAP2, | squamous epithelium, cervix, cervix, |
| | GTPBP2, ANGPTL2, CASP3, | chorion, chorionic villi, decidua, decidua, |
| | CA13, DNAJB6, SERPINA5, | ectocervical epithelium, embryo testis, |
| | ABHD14B, GMPR2, CCL26, | endometrium, endometrium, |
| | ADAMTSL4, PLSCR3 | endometrium basalis, endometrium |
| | | epithelium, epididymis, epididymis, |
| | | epididymis lumen, excurrent ducts, |
| | | fallopian tube, fallopian tubes, fallopian |
| | | tubes, female reproductive, fetal testis, |
| | | fetal testis, foreskin, genital, genital |
| | | tract, gingival crevicular, gingival |
| | | crevicular fluid, gonadal ridge, graaf |
| | | follicle fluids, isthmus, mammary, |
| | | mammary epithelia, mammary epithelial |
| | | cell surfaces, mammary gland, mammary |
| | | gland, mammary glands, myometrium, |
| | | neoplastic prostate, nipple aspirate, |
| | | nipple epidermis, outer myometrial |
| | | smooth muscle, ovarian, ovarian, ovaries, |
| | | ovary, ovary, oviduct, penis, prostate, |
| | | prostate, prostate, prostate epithelium, |
| | | prostate gland, prostate gland, prostate |
| | | glands, prostatic, prostrate, |
| | | reproductive, reproductive system, |
| | | seminal vesicle, seminal vesicle, seminal |
| | | vesicles, seminiferous tubules, testes, |
| | | testicles, testis, testis, umbilical chord, |
| | | umbilical cord, uterine endometrium, |
| | | uterine fluid, uterine glandular |
| | | epithelium, uterine myometrium, uterus, |
| | | vagina, vaginal epithelium |
| Respiratory | VSIR, BDNF, FRZB, EREG, | airway epithelium, airways, alveolar |
| | CASP2, PLXNB3, BID, PEAR1, | walls, bronchi, bronchial epithelial, |
| | PRKG1, CCL13, EDAR, GTPBP2, | bronchial glands, bronchial submucosal, |
| | PDLIM7, DRG2, CASP3, | bronchiolar epithelium, bronchioles, |
| | DNjAJB6, MAX, ABHD14B, | bronchus, bronchus, bronchus- |
| | CCL17, STX8, ADAMTSL4, | associated, fetal lung, fetal lung, larynx, |
| | PLSCR3 | lung, lung, lung endothelium, lung |
| | | parenchyma, lung submucosal, lung |
| | | submucosal gland acinus, lung vascular |
| | | smooth muscle, lungs, nasal, nasal cavity, |
| | | nasal mucosa, nasal septal epithelium, |
| | | nasopharynx, pharynx, pulmonary, |
| | | pulmonary airways, pulmonary alveoli, |
| | | respiratory epithelium, respiratory tracts, |
| | | ribs, trachea, trachea |
| Urinary | EGF, APP, FRZB, EREG, CASP2, | ascending limbs, bladder, bladder, |
| | TBC1D23, SEPTIN9, PLXNB3, | bladder urothelium, collecting duct, |
| | ADAMTS15, PEAR1, ENOX2, | collecting ducts, collecting tubule, |
| | EDAR, DRG2, GTPBP2, VPS37A, | convoluted tubule, convoluted tubule, |
| | CASP3, DNAJB6, ERBIN, MAX, | convoluted tubule lumen, convoluted |
| | SERPINA5, ABHD14B, GMPR2, | tubules, cortical collecting tubules, |
| | STX8, ADAMTSL4, PLSCR3 | descending limbs, distal tubules, fetal |
| | | bladder, fetal kidney, fetal kidney, fetal |
| | | kidney, fetal kidneys, glomeruli, |
| | | glomerulus, henle, kidney, kidney, kidney |
| | | artery, kidney cortex, kidney distal, |
| | | kidney glomeruli, kidney medulla, |
| | | kidneys, mesangium, nephron, nephron |
| | | segments, non tumor kidney, normal |
| | | kidney, proximal tubule, proximal |
| | | tubules, renal proximal tubule, renal |
| | | proximal tubules, ureter, ureter, urinary |
| | | bladder, urinary bladder, urogenital, |
| | | urogenital, urothelium, vas deferens |

Note:
The proteins in Table 5 are listed in ascending Bonferroni adjusted P-Value order.

TABLE 6
Expression NLP Categories by Cell Type for the Top 119 Proteins

| Cell Type | Proteins | Keywords |
|---|---|---|
| Adipocyte | C3 | adipocyte, adipocytes |
| Cancer | GIPC3, ENOX2, | 3, a-431 epidermoid carcinoma, a-549 (lung |
| | SERPINA5 | carcinoma), acute myelocytic leukemia, acute |
| | | myelogenous leukemia), acute myeloid leukemia, |
| | | all leukemia/lymphoma lines, bladder cancer, |
| | | bladder carcinoma, breast cancer, breast cancer |
| | | lines, breast cancer lines mcf-7, breast cancer |
| | | lines mda-mb-231, breast cancer lines, breast |
| | | carcinoma lines, burkitt's lymphoma lines, |
| | | cancer, cancer lines, carcinoma, carcinoma lines, |
| | | choriocarcinoma, choriocarcinoma cancer lines, |
| | | colon adenocarcinoma line t84, colon |
| | | adenocarcinoma lines, colon cancer lines, |
| | | colorectal adenocarcinoma line, colorectal |
| | | cancer, colorectal cancer lines, colorectal tumor, |
| | | erythroleukemia, erythroleukemia line k-562, |
| | | fa6, fibrosarcoma, gastric, gastric cancer lines, |
| | | glioblastoma, glioblastoma lines, glioblastomas, |
| | | hairy leukemia, hbl-100 breast carcinoma, hel, |
| | | hematopoietic tumor lines, hepatoular |
| | | carcinoma, hl-60, hodgkin, hpaf, hs 294t |
| | | melanoma, ht29-d4 colon carcinoma, imim-pc2, |
| | | intratumoral nk, k-562, k-562 erythroleukemia, |
| | | kidney tumor, leiomyomal, leukemia lines, |
| | | leukemia u-937, leukemia u-937 line, leukemic, |
| | | leukemic lines, lovo, lung cancer lines, lung |
| | | carcinoma lines, lung tumor lines, lymphoma, |
| | | lymphoma lines, malignant, malignant hodgkin |
| | | lymphoma, malignant melanoma, malignant |
| | | melanoma lines, mammary carcinoma lines, |
| | | mcf-7 breast carcinoma, mda-mb-175, mda-mb- |
| | | 435, melanoma, melanoma lines, metastasizing |
| | | melanoma lines, myelogenous leukemia line kg- |
| | | 1, myelogenous leukemic lines, myeloid leukemia |
| | | lines, nb4, neoplastic lines, neuro-epithelioma, |
| | | neuroblastoma, non invasive breast carcinoma |
| | | lines, non-glial-derived nervous system tumor |
| | | lines, non-hodgkin lymphoma lines, |
| | | noneuroblastoma, nonhematopoietic tumor |
| | | lines, nurse-like, pancreatic cancer lines, |
| | | pancreatic carcinoma lines, panctu-ii, |
| | | paraneoplastic tumor, pc-3, promyelocytic |
| | | leukemia line hl-60, prostate cancer, prostate |
| | | cancer lines, prostatic adenocarcinoma lines, |
| | | retinoblastoma lines, several cancer lines, sk-ov- |
| | | 3 (ovary adenocarcinoma), smmc7721, snu-c2b |
| | | colon carcinoma, sw48, sw480, sw480 colon |
| | | carcinoma, sw480 colorectal cancer line, |
| | | testicular tumor, tumor, tumor lines, tumor |
| | | endothelial, tumor invasive tumors, tumor- |
| | | derived lines, tumoral, tumorigenic lines, tumors |
| | | lines, u-251mg, u-937 histiocytic lymphoma lines |
| Chondrocyte | | articular chondrocytes, chondrocyte, |
| | | chondrocyte-like, chondrocytes, fetal |
| | | chondrocyte |
| Dendritic | CD84 | cutaneous dendritic, dc, dendritic, follicular |
| | | dendritic, ikdcs, imddc, immature dendritic, |
| | | interdigitating reticulum, interferon-producing |
| | | killer dendritic, mddc, monocyte-derived, |
| | | monocyte-derived dendritic, myeloid blood |
| | | dendritic, myeloid dendritic, pdc, pdcs, peripheral |
| | | blood plasmacytoid dendritic, plasmacytoid, |
| | | plasmacytoid blood dendritic, plasmacytoid |
| | | dendritic, plasmacytoids, thymic dendritic, |
| | | tolerogenic dcs, tonsil dc, tonsil interdigitating |
| | | dendritic, various dendritic |
| Dental | | ameloblast, cementoblast, odontoblast, |
| | | ondotoblasts |
| Endocrine | | endocrine, enterocyte-like, enterocytes, |
| | | enteroendocrine, enteroendocrine I, ileal |
| | | absorptive enterocytes |
| Endothelial | SELP, PEAR1, | angioblasts, aorta endothelial, aortic endothelial, |
| | CRACR2A | arterial endothelial, artery endothelial, blood |
| | | brain barrier endothelial, cervical epithelium, |
| | | umbilical vein endothelial, endiothelial, |
| | | endothelial, endothelial venules, human umbilical |
| | | vein endothelial, huvecs, liver sinusoidal |
| | | endothelial, lung endothelial, lymph vessel |
| | | endothelial, microvascular capillary endothelial, |
| | | microvessels endothelial, placenta, umbilical vein |
| | | endothelial, renal glomeruli endothelial, |
| | | reticuloendothelial, sinusoidal endothelial, tumor |
| | | endothelial, umbilical veil endothelial, umbilical |
| | | vein endothelial, vascular endothelial |
| Epithelial | FN1, TMEM106A, | a-549, airway epithelial, alveolar epithelial, |
| | ARL2BP, SDC4 | alveolar type 2, alveolar type ii, antral epithelial, |
| | | atypical epithelial, breast epithelial, breast |
| | | epithelial line mcf-10a, breast epithelial lines, |
| | | bronchial, bronchial epithelial, choroid plexus |
| | | epithelial, ciliary body epithelial, ciliated, colonic, |
| | | colonic epithelial, columnar epithelial, corneal |
| | | epithelial, embryonic epithelial, epithelial, |
| | | epithelial lines, eue, gastric epithelial, |
| | | gastrointestinal epithelial, hek293, hela, helas3, |
| | | hep-g2, ht-29 colonic epithelial, intestinal |
| | | epithelial, intraepithelial cd8-positive t, |
| | | intraepithelial lymphocytes, kidney epithelial, |
| | | kidney proximal tubular epithelial, luminal |
| | | epithelial, lung alveolar type 2, lung epithelial, |
| | | mammary epithelial, mcf-7, mesothelial, |
| | | myoepithelial, myoepithelium, nasal, nasal |
| | | epithelial, nonciliated, paneth, parietal epithelial, |
| | | pharyngeal epithelial, prostate gland epithelial, |
| | | renal proximal tubule epithelial, retina pigment |
| | | epithelial, retinal pigment epithelial, secretory |
| | | epithelial, small intestinal epithelial, surface |
| | | epithelial, t-47d, thymic, thymic epithelial, |
| | | thyrocytes, tracheal surface epithelial, tubular |
| | | epithelial, type ii alveolar, unpolarized epithelial, |
| | | vascular epithelial, zr-75-1 |
| Erythrocyte | PEAR1 | erythroblasts, erythrocytes, erythroid, fetal |
| | | erythrocytes |
| Eye | | amacrine, cone, photoreceptors, rod |
| | | photoreceptor |
| Fibroblast | FN1, CHMP1A, | cerebral pericytes, dermal fibroblasts, fetal |
| | LTA4H, HEPH, SDC4 | fibroblasts, fibroblast, fibroblast lines, fibroblast |
| | | lines tk, fibroblast-like synoviocytes, fibroblastic, |
| | | fibroblasts, foreskin fibroblast, foreskin |
| | | fibroblasts, gingival fibroblasts, mg-63 line, |
| | | myofibroblasts, pulmonary fibroblasts, skin |
| | | fibroblasts, stromal fibroblast, stromal |
| | | fibroblasts, synovial, synovial fibroblasts, synovial |
| | | fluid |
| Glial | APP, PLXNB3 | astrocytes, astrocytoma, glia, glial, glioma, glioma |
| | | lines, glioma tissue, liver astrocytes, microglia, |
| | | microglial, neuro-glial, olgs, oligodendrocytes, |
| | | oligodendroglia, perivascular astrocytes |
| Granulocyte | CD69, LTA4H, | basophil, basophils, bone marrow neutrophils, |
| | PLXNB3 | eosinophils, granular, granule, granulocyte, |
| | | granulocytes, granulocytic, granulocytic lineage, |
| | | inflammatory, neutrophil, neutrophil lineage, |
| | | neutrophils, peripheral blood basophils, |
| | | peripheral blood granulocytes, peripheral blood |
| | | neutrophils, polynuclear neutrophils |
| Hematopoietic | | hematopoetic, hematopoietic, hematopoietic |
| | | lines, hematopoietic lineage, hematopoietic |
| | | precursors, hematopoietic progenitor, |
| | | hematopoietic stem, hemopoietic |
| Kidney | TMEM106A, | bowman's capsule, distal tubular, embryonal |
| | PLXNB3 | kidney, embryonic kidney, glomerular epithelium, |
| | | glomerular mesangial, hsc, interstitial, kidney |
| | | distal tubular, mesangial, podocyte, podocytes, |
| | | proximal tubule, renal, renal lines, renal proximal |
| | | tubular, tubular |
| Leukocytes Nyd | SKAP1, EREG, | ag-presenting, apcs, blood leukocytes, blood |
| | CD84, HEPH, | mononuclear, bone, bone marrow, bone marrow |
| | PEAR1, NFATC1, | mononuclear, bone marrow-derived, bone |
| | PDLIM7, DRG2, | marrow-derived mesenchymal stem, bone |
| | DNAJB6, ABHD14B, | trabecular, bone-derived, cortical thymocytes, |
| | GMPR2, | flattened bone-lining, immune, immunoblasts, |
| | ADAMTSL4 | immunocyte lines, interstitial leukocytes, |
| | | leukocyte, leukocyte lines, leukocytes, mast, |
| | | mononuclear, mononuclear leukocytes, |
| | | myeloblast, normal mast, pbmc, pbmcs, |
| | | peripheral blood leukocyte, peripheral blood |
| | | leukocytes, peripheral blood mononuclear, |
| | | peripheral blood mononuclear leukocytes, |
| | | peripheral leukocyte, peripheral leukocytes, |
| | | peripheral mononuclear, phagocytes, phagocytic, |
| | | plasma, pmns, polymorphonuclear leukocytes, |
| | | polynuclear, promyelocyte stage, promyelocytes, |
| | | promyelocytic, submucosal leukocytes, |
| | | thymocytes, urothelial, white blood |
| Liver | FN1 | crypt, hepatic, hepatic parenchymal, hepatic |
| | | stellate, hepatocytes, hepatoma, hepatoma lines, |
| | | liver hepatocytes |
| Lymphocyte | VSIR, SKAP1, APP, | -differentiated hl-60, alpha-beta t, b, b lineage, b-, |
| | CD69, BIN2, LTA4H, | b- lineage, b- lines, b--like line raji, b-1, b- |
| | CD84, CCL5, | lymphocyte, b-lymphocytes, b-lymphoid lines, b- |
| | NFATC1, CD226, | lymphomas, blood lymphocytes, ca4, cytolytic, |
| | DGKA, PLSCR3, | cytotoxic t lymphocytes, cytotoxic t-lymphocytes, |
| | CRACR2A | decidual nk, effector, epstein-barr virus- |
| | | transformed lymphoblastoid lines, fetal nk-, |
| | | gamma delta t, gamma-delta t, gamma-delta t-, |
| | | germinal center, germinal center b-, germinal |
| | | centers, group2 innate lymphoid, helper t-, hsb, |
| | | hut 78, ilc2s, intraepithelial lymphocytes, |
| | | intratumoral nk, jurkat, jurkat lines, jurkat t- |
| | | leukemia, jurkat t- line, large lymphocytes, |
| | | lymphoblast, lymphoblasts, lymphocyte, |
| | | lymphocytes, lymphocytic lines, lymphocytic |
| | | lineage, lymphoid, lymphoid lines, lymphoid |
| | | organs, marginal zone b-, mature b, melanoma- |
| | | specific cytotoxic t clones, memory b-, memory |
| | | gamma-delta t, memory t-, memory th17, molt-4, |
| | | molt-4 lines, naive t, natural killer, natural killer |
| | | lines nkl, natural killer (nk), neoplastic b- and t- |
| | | lines, neoplastic b- and t- lines, nk, nk subsets, nk |
| | | 62, nk-, nk- line, nkt, normal germinal center (gc) |
| | | b-, pbl, peripheral blood lymphocyte, peripheral |
| | | blood lymphocytes, peripheral blood memory t-, |
| | | peripheral blood t-, peripheral blood t- |
| | | lymphocytes, peripheral lymphocytes, peripheral |
| | | memory, peripheral t-, peripherical blood |
| | | lymphocytes, plasma b-, pre b-, pre t-, pre-b-, |
| | | pro-b precursors, raji b-lymphoblasts, reed- |
| | | sternberg (hrs), sup-t1, t, t populations, t |
| | | lymphocytes, t-, t- clones, t- leukemia lines molt- |
| | | 4, t- lineage, t- lines, t- lines harris, t- lymphoid |
| | | lines, t- subsets, t-helper, t-helper 2, t- |
| | | lymphoblasts, t-lymphocytes, th0, th1, thymus- |
| | | derived t-, tonsillar germinal center centrocytes, |
| | | transitional b, treg, treg), yt |
| Macrophage | CD69, CD84, | -macrophage, a, alveolar macrophages, bone |
| | PEAR1, CCL5 | marrow macrophages, cd68, cortical |
| | | macrophages, decidual macrophages, epidermal |
| | | langerhans, epidermoid, hofbauer, kg-1, kupffer, |
| | | langerhans, langerhans', liver kupffer, liver |
| | | kupffer, lung alveolar macrophages, m1 |
| | | macrophages, macrophage, macrophage line, |
| | | macrophage lines u-937, macrophage |
| | | progenitor, macrophage-like, macrophages, |
| | | meningeal macrophages, monocyte-derived |
| | | macrophages, monocyte-derived macrophages, |
| | | non sec-, perivascular macrophages, placental |
| | | macrophages, red pulp macrophages, spleen |
| | | macrophage, tissue macrophages |
| Monocytes | VSIR, LTA4H, CD84 | apcs monocytes, cd11b monocytes, mono-mac-6, |
| | | monocyte, monocyte-like line u-937, monocyte- |
| | | related, monocytes, monocytic, monocytic lines, |
| | | myelomonocytic, myelomonocytic lineage, |
| | | peripheral blood monocytes, peripheral |
| | | monocytes, promonocytic, thp-1, thp-1 |
| | | monocytes |
| Mucous | | bronchial goblet, ciliated bronchiolar, goblet, |
| | | intestinal mucosa, mucous, mucus, mucus- |
| | | secreting, nasal goblet secretory, upper gastric |
| | | mucosal |
| Muscle | PEAR1, CCL13 | airway smooth muscle, aortic smooth muscle, |
| | | arterial smooth muscle, artery smooth muscle, |
| | | cardiac, cardiac myocytes, cardiomyocytes, |
| | | cerebral artery smooth muscle, coronary muscle, |
| | | muscle, myoblasts, myocytes, myotubes, |
| | | perivascular, placental vascular smooth muscle, |
| | | placental villi smooth muscle, pulmonary artery |
| | | smooth muscle, skeletal muscle, smooth, smooth |
| | | muscle, umbilical vein smooth muscle, vascular, |
| | | vascular smooth, vascular smooth muscle, |
| | | vascular wall, vsmc |
| Myeloid | VSIR, GIPC3 | chronic myelogenous, early myeloid lines, |
| | | monocytic/myeloid lineage, mucosal myeloid, |
| | | myeloid, myeloid lines, myeloid lineage, myeloid |
| | | lineages, myeloid progenitor, myeloid-derived |
| | | suppressor, myeloids, myeloma, myeloma line |
| | | u266b1, myeloma line u266r |
| Neuron | SRC, PLXNB3, | axons, basket, brain neurons, ca2, central |
| | PEAR1, CASP3, | neurons, cerebellar purkinje, cerebral cortex, |
| | ENO2 | cortical neurons, dentate gyrus granule neurons, |
| | | dopaminergic neurons, dorsal root ganglia |
| | | neurons, dorsal root ganglion, gabaergic neurons, |
| | | granule neurons, gray matter neurons, |
| | | hippocampal pyramidal neurons, hippocampus |
| | | pyramidal, hippocampus pyramidal neurons, |
| | | lines, motoneurons, neocortical neurons, neural, |
| | | neural crest, neural progenitor, neural stem, |
| | | neuroendocrine, neuroendocrine epithelium, |
| | | neuron, neuronal, neurons, olfactory receptor |
| | | neurons, peripheral, peripheral neurons, pontine |
| | | nuclei, purkinje, purkinje neurons, pyramidal, |
| | | pyramidal neurons, retinal ganglion, schwann, |
| | | schwann culture, somatomotor, spinal cord |
| | | neurons, stellate, striatal neurons |
| Non-Hematopoietic | | non-hematopoetic, non-hematopoietic, |
| | | nonhematopoietic |
| Osteoblast | CHMP1A, SRC, | bone osteoblasts, giant osteoclast-like, |
| | PEAR1 | osteoblast, osteoblast line mg-63, osteoblast line |
| | | saos-2, osteoblast-like, osteoblasts, osteoclasts, |
| | | osteocytes, osteogenic, osteosarcoma lines, |
| | | primary ossification center-associated, |
| | | subchondral bone osteoblasts |
| Other | APP | epidermal basal, inner ear hair, intimal, large t |
| | | sv40 antigen-, non-immune, non-neuronal, |
| | | nonmuscle, normal lines, populations, villous |
| Other Blood | NT5C3A, LTA4H | blood, blood lines, cord blood, peripheral blood, |
| | | peripherical blood, red blood, reticulocytes |
| Pancreatic | | beta-, betaacinar, ductile, islet, pancreas islet |
| | | beta, pancreatic, pancreatic lines, pancreatic |
| | | acinar, pancreatic beta, pancreatic islet |
| Platelet | GP6, GP5, SELP, | eosinophil platelets, megakaryoblastic, |
| | D69, TREML1, SRC, | megakaryocytes, megakaryocytic, platelet, |
| | LTA4H, CD84, | platelets, thrombocytes |
| | PEAR1, VAMP8, | |
| | SERPINA5 | |
| Reproductive | DNAJB6, SERPINA5 | cumulus, cyto- and syncytiotrophoblastic, |
| | | cytotrophoblasts, endometrial glandular, |
| | | extravillous trophoblast, germ, gonocytes, |
| | | granulosa, interstitial leydig, leydig, luteinized |
| | | granulosa, migratory primordial germ, |
| | | myometrial, oocytes, ovarian granulosa, ovary, |
| | | post-meiotic, postnatal leydig, secretory |
| | | endometrial, sertoli, spermatocytes, |
| | | spermatogenic, spermatogonia, spermatogonias, |
| | | spermatozoa, syncytiotrophoblast, |
| | | syncytiotrophoblasts, testicular somatic, |
| | | trophoblast, trophoblasts, x |
| Secretory | SELP | chief, chromaffin, clara, enterochromaffin, fundic, |
| | | gastric parietal, secretory, serous-like, weibel- |
| | | palade bodies, zymogen-producing |
| Skin | VAV3, EDAR, | basal keratinocytes, cornified, epidermal basal |
| | SERPINA5 | layer keratinocytes, epidermal keratinocytes, |
| | | keratinocyte, keratinocytes, megakaryocytic |
| | | lines, melanocyte, melanocytes, pigment, skin |
| | | keratinocytes, suprabasal keratinocytes |
| Spleen | SKAP1 | fetal spleen, spleen, splenic marginal zone, |
| | | splenocytes |
| Stem | | cambial, cml) stem, embryonic stem, esc, |
| | | mesenchymal, mesenchymal stem, stem |
| Stromal | | bone marrow stromal, endometrial stromal, non- |
| | | cancerous stromal, stromal, stromal type |

Note:
The proteins in table 6 are listed in ascending Bonferroni adjusted P-Value order. Keywords and cell types are generated from all proteins with available information. Table 6 displays the top 119 proteins. Some cell types listed were not linked to any of the 119 proteins (e.g., Pancreatic).

### REFERENCES

1. Harrison A G, Lin T, Wang P. Mechanisms of SARS-CoV-2 Transmission and Pathogenesis. Trends in Immunology. 2020; 41(12):1100-15.
2. Yuki K, Fujiogi M, Koutsogiannaki S. COVID-19 pathophysiology: A review. Clinical Immunology. 2020; 215:108427.
3. Koyama S, Ishii K J, Coban C, Akira S. Innate immune response to viral infection. Cytokine. 2008; 43(3):336-41.
4. Garvin M R, Alvarez C, Miller J I, Prates E T, Walker A M, Amos B K, et al. A mechanistic model and therapeutic interventions for COVID-19 involving a RAS-mediated bradykinin storm. Elife. 2020; 9.
5. Fraser D D, Cepinskas G, Slessarev M, Martin C, Daley M, Miller M R, et al. Inflammation Profiling of Critically Ill Coronavirus Disease 2019 Patients. Crit Care Explor. 2020; 2(6):e0144.
6. Gill S E, dos Santos C C, O'Gorman D B, Carter D E, Patterson E K, Slessarev M, et al. Transcriptional profiling of leukocytes in critically ill COVID19 patients: implications for interferon response and coagulation. Intensive Care Medicine Experimental. 2020; 8(1):75.
7. Juneja G K, Castelo M, Yeh C H, Cerroni S E, Hansen B E, Chessum J E, et al. Biomarkers of coagulation, endothelial function and fibrinolysis in critically-ill patients with COVID-19: A single-centre prospective longitudinal study. J Thromb Haemost. 2021.
8. Cani E, Dwivedi D J, Liaw K-L, Fraser D D, Yeh C H, Martin C, et al. Immunothrombosis Biomarkers for Distinguishing Coronavirus Disease 2019 Patients From Noncoronavirus Disease Septic Patients With Pneumonia and for Predicting ICU Mortality. Critical Care Explorations. 2021; 3(12).
9. Fraser D D, Patterson E K, Slessarev M, Gill S E, Martin C, Daley M, et al. Endothelial Injury and Glycocalyx Degradation in Critically Ill Coronavirus Disease 2019 Patients: Implications for Microvascular Platelet Aggregation. Crit Care Explor. 2020; 2(9):e0194.
10. Ackermann M, Verleden S E, Kuehnel M, Haverich A, Welte T, Laenger F, et al. Pulmonary Vascular Endothelialitis, Thrombosis, and Angiogenesis in Covid-19. N Engl J Med. 2020.
11. Bertsimas D, Lukin G, Mingardi L, Nohadani O, Orfanoudaki A, Stellato B, et al. COVID-19 mortality risk assessment: An international multi-center study. PLOS ONE. 2020; 15(12):e0243262.
12. Raveendran A V, Jayadevan R, Sashidharan S. Long COVID: An overview. Diabetes & Metabolic Syndrome: Clinical Research & Reviews. 2021; 15(3):869-75.
13. Crook H, Raza S, Nowell J, Young M, Edison P. Long covid-mechanisms, risk factors, and management. BMJ. 2021:n1648.
14. Davis H E, Assaf G S, McCorkell L, Wei H, Low R J, Re'Em Y, et al. Characterizing long COVID in an international cohort: 7 months of symptoms and their impact. EClinicalMedicine. 2021; 38:101019.
15. Huang C, Huang L, Wang Y, Li X, Ren L, Gu X, et al. 6-month consequences of COVID-19 in patients discharged from hospital: a cohort study. The Lancet. 2021; 397(10270):220-32.
16. Salamanna F, Maglio M, Landini M P, Fini M. Body Localization of ACE-2: On the Trail of the Keyhole of SARS-CoV-2. Front Med (Lausanne). 2020; 7:594495.
17. Gavriilaki E, Eftychidis I, Papassotiriou I. Update on endothelial dysfunction in COVID-19: severe disease, long COVID-19 and pediatric characteristics. Journal of Laboratory Medicine. 2021; 45(6):293-302.
18. Matta J, Wiernik E, Robineau O, Carrat F, Touvier M, Severi G, et al. Association of Self-reported COVID-19 Infection and SARS-CoV-2 Serology Test Results With Persistent Physical Symptoms Among French Adults During the COVID-19 Pandemic. JAMA Internal Medicine. 2022; 182(1):19-25.
19. NIH HRPP. POLICY: Guidelines for Limits of Blood Drawn for Research Purposes in the Clinical Center. M95-9 (rev.) 2009; 5.
20. Brisson A R, Matsui D, Rieder M J, Fraser D D. Translational research in pediatrics: tissue sampling and biobanking. Pediatrics. 2012; 129:153-62.
21. Gillio-Meina C, Cepinskas G, Cecchini E L, Fraser D D. Translational research in pediatrics II: blood collection, processing, shipping, and storage. Pediatrics. 2013; 131:754-66.
22. Priestap F, Kao R, Martin C M. External validation of a prognostic model for intensive care unit mortality: a retrospective study using the Ontario Critical Care Information System. Can J Anaesth. 2020; 67:981-91.
23. Singer M, et al. The third international consensus definitions for sepsis and septic shock (Sepsis-3). JAMA. 2016; 315:801-10.
24. Lundberg M, Eriksson A, Tran B, Assarsson E, Fredriksson S. Homogeneous antibody-based proximity extension assays provide sensitive and specific detection of low-abundant proteins in human blood. Nucleic Acids Res. 2011; 39:e102.
25. Assarsson E, et al. Homogenous 96-plex PEA immunoassay exhibiting high sensitivity, specificity, and excellent scalability. PLoS ONE. 2014; 9: e95192.
26. Bradley A P. The use of the area under the ROC curve in the evaluation of machine learning algorithms. Pattern Recogn. 1997; 30:1145-59.
27. Van der Maaten L, Hinton G. Visualizing data using t-SNE. J Mach Learn Res. 2008; 9:11.
28. Jambu M. Chapter 10-classification of individuals-variables data sets. In: Jambu M, editor. Exploratory and multivariate data analysis. Boston: Academic Press; 1991. p. 305-405.
29. Pedregosa F, et al. Scikit-learn: machine learning in Python. J Mach Learn Res. 2011; 12:2825-30.
30. Bateman A, et al. UniProt: the universal protein knowledgebase in 2021. Nucleic Acids Res. 2021; 49:D480-9.
31. Zhang Y, Zhang Y, Qi P, Manning C D, Langlotz C P. Biomedical and clinical English model packages for the Stanza Python NLP library. J Am Med Inform Assoc. 2021a; 28:1892-9.
32. Qi P, Zhang Y, Zhang Y, Bolton J, Manning C D. Stanza: a Python natural language processing toolkit for many human languages. 2020. arXiv: 2003. 07082.
33. Honnibal M, Montani I, Van Landeghem S, Boyd A. SpaCy: Industrial-strength Natural Language Processing in Python; 2020.

Through the embodiments that are illustrated and described, the currently contemplated best mode of making and using the invention is described. Without further elaboration, it is believed that one of ordinary skill in the art can, based on the description presented herein, utilize the present invention to the full extent. All publications cited herein are incorporated by reference.

Although the description above contains many specificities, these should not be construed as limiting the scope of the invention, but as merely providing illustrations of some of the presently embodiments of this disclosure.

1. A method of diagnosing long-COVID-19 in a patient, the method comprising: (a) obtaining a test sample from the patient, (b) performing one or more assays configured to detect a level of one or more biomarkers in the test sample, (c) comparing the level of the one or more proteins in the test sample with a healthy control reference value of said one or more proteins, wherein a change in the level of the one or more biomarkers in the test sample relative to the healthy control reference value of said one or more proteins is indicative of long-COVID-19 diagnosis, wherein the one or more proteins are selected from Table 3.

2. The method of claim 1, wherein the one or more proteins are selected from CXCL5, AP3S2, MAX, PDLIM7, ED AR, LTA4H, CRACR2A, CXCL3, FRZB.

3. The method of claim 1, wherein the one or more proteins are selected from CXCL5, AP3SE, MAX, PDLIM7, and FRZB.

4. The method of claim 1, wherein said one or more assays is a proteomic assay.

5. The method of claim 1, wherein when the patient is diagnosed with long-COVID-19, the method further includes treating the patient with a long-COVID-19 therapy.

6. The method of claim 5, wherein said long-COVID-19 therapy comprises administering to the patient a treatment that promotes angiogenesis and/or administering to the patient accelerators of angiogenesis.

7. The method of claim 5, wherein said long-COVID-19 therapy comprises administering to the patient at least one of the proteins listed in Table 3 having its level lower than the level of said one or more biomarker in the healthy control and/or acute COVID-19 reference value.

8. The method of claim 5, wherein said long-COVID-19 therapy comprises administering to the patient at least one of FRZB, a source of FRZB, FN1, a source of FN1, CKMT1A, a source of CKMT1A, CKMT1B, a source of CKMT1B, HS6ST1, a source of HS6ST1, BMP6, a source of BMP6, ADAMTS15, a source of AD AMTS 15, ANGPTL2, a source or ANGPTL2, IFNLR1, C1QA, a source of C1QA, DRAXIN, a source of DRAXIN, ADAMTSL and/or a source of ADAMTSL4.

9. The method of claim 5, wherein said long-COVID-19 therapy comprises administering to the patient an agent that reduces the level of at least one of the proteins listed in Table 3 having its level higher than the level of said one or more proteins in the healthy control reference value.

10. The method of claim 5, wherein said long-COVID-19 therapy comprises administering to the patient an agent that reduces the levels of at least one of CXCL5, LTA4H, CXCL3, ED AR, MAX, PDLIM7, CRACR2A and AP3S2.

11. The method of claim 1, wherein the method further comprises (i) obtaining one or more recovery samples from the subject during the subject's treatment for long-COVID, and (ii) comparing the level of at least one of the proteins listed in Table 3 in the recovery samples to the level of said at least one protein in the test sample and to the level of said at least one protein in the healthy control reference value, wherein an approximation in the levels of the at least one protein in the one or more recovery samples towards the healthy control reference value for said at least one protein relative to the levels of the one or more biomarker obtained in the test sample is indicative of a normalization of the patient.

12. A method of treating long-COVID-19 in a patient, the method comprising administering to the patient at least one of: (i) one or more of the proteins listed in Table 3 having a level in the long-COVID-19 column lower than the level of said one or more proteins in the Healthy and CO VID column, and (ii) an agent that reduces the level of one or more of the proteins listed in Table 3 having a level in the long-COVID column of Table 3 higher than the level of said one or more proteins in the Healthy and CO VID column of Table 3.

13. The method of claim 12, wherein the agent reduces the level of one or more of CXCL5, LTA4H, CXCL3, ED AR, MAX, PDLIM7, CRACR2A and AP3S2.

14. The method of claim 12, wherein the method comprises administering to the patient at least one protein is FRZB, a source of FRZB, FN1, a source of FN1, CKMT1A, a source of CKMT1A, CKMT1B, a source of CKMT1B, HS6ST1, a source of HS6ST1, BMP6, a source of BMP6, ADAMTS15, a source of AD AMTS 15, ANGPTL2, a source or ANGPTL2, IFNLR1, C1QA, a source of C1 QA, DRAXIN, a source of DRAXIN, ADAMTSL and/or a source of ADAMTSL4.

15-18. (canceled)
